SEQUENCE LISTING (15 sequences)

SEQ ID NO: 1 - DNA, 2974 bp, Homo sapiens
  CDS (115)..(2220)
  sig_peptide (115)..(216)
  mat_peptide (217)..(2157)
  sig_peptide (2158)..(2220)
ctcagggcagagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggattgctgattctttaaatgtcttgtttcccagatttcaggaaactttttttcttttaagctatccactcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtataacagaaaaa

SEQ ID NO: 2 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 3 - DNA, 2059 bp, Artificial sequence
  Human CEA-encoding DNA sequence codon optimized for non-human expression
gctagccaccatggaaagtccctcagccccaccccaccgctggtgtattccttggcagcgcctgctcctgacagcaagcctgctgaccttttggaatccacccacgacagccaaactgactatcgagtctactcctttcaacgtggcggaagggaaggaggttctgctcctggtccataatctgccacagcacctgtttggttattcatggtacaaaggcgaacgcgtggatggaaaccggcaaattatcgggtatgttatcggcacacagcaggccacacccggtccggcttacagtgggcgcgaaattatctatccaaatgccagcctgctgatccaaaacattatccagaatgacactggcttctacacactgcatgtcatcaaaagcgatctggtgaacgaggaagcaacgggtcagtttcgggtttatcccgaactgcctaagccatcaatttcttccaataacagtaaacccgtcgaagacaaagacgccgtggctttcacgtgcgagcctgaaacacaagatgcaacttacctgtggtgggttaacaatcagagcctgccagtctcaccccgcctgcagctgagcaacggaaatcggaccctgacactctttaacgtgacgcgcaatgacactgcctcttataagtgtgagacacaaaaccctgtttcagcgcggcgcagcgatagtgttatcctgaatgtgctgtacggcccagacgctcccaccatttccccgctgaacacttcttatcgctcaggggaaaatctgaacctgagctgccacgccgcaagcaatccacccgcgcagtacagttggttcgtcaacggtacatttcagcaatctactcaggaactgttcatccctaatattacggttaacaattcaggcagctatacatgtcaggctcataactccgacacagggctgaatcggactacagtgacgacgatcacagtctacgccgagccacccaaaccctttattacttctaacaatagtaacccagttgaagatgaggacgcagtggctctgacctgcgaacctgagatccaaaatacaacctatctgtggtgggtcaacaatcagtcactgccagttagcccccgcctgcagctcagcaacgacaatcggactctgacgctgctctctgtgacacgcaacgatgtcggtccatacgaatgtggcattcaaaatgaactgtcagttgaccacagtgatcccgtgatcctgaacgtcctgtatggaccagacgaccctaccattagcccatcctacacttattaccggcccggggttaatctgtctctgagctgccatgccgcgtcaaacccacccgcacagtacagctggctgatcgatggcaatattcagcaacacacacaggagctgttcatcagtaacattacagaaaaaaattctggactgtatacgtgtcaggctaacaattccgccagcggtcatagccgcacaactgtgaagacgatcactgtctctgccgagctgccgaaaccaagtatttcatccaacaatagcaagcccgttgaagacaaagatgcagtggcctttacatgcgagccagaagctcaaaacactacgtacctgtggtgggtcaatggccagtctctgcccgttagcccacggctccagctgagtaacgggaatcgcacactgaccctgttcaacgtgacccggaatgacgcgcgcgcctatgtctgtggaatccaaaactccgttagcgcaaatcggtctgaccctgtgacactggatgtcctgtacggcccagacactcccattatctcaccacccgatagcagttatctgagcggtgctaacctgaatctctcttgccactccgcctcaaacccttctccacagtacagctggcgcatcaatgggattccccagcaacatacgcaggttctgtttatcgcaaaaattacaccaaacaataacggcacctatgcgtgtttcgtgagtaacctggccactggacggaataacagcatcgtcaagtccattacagtttctgcaagcggtacgtcaccctaatga

SEQ ID NO: 4 - protein, 681 aa, Artificial sequence
  Human CEA-encoding DNA sequence codon optimized for non-human expression
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSP

SEQ ID NO: 5 - DNA, 2167 bp, Artificial sequence
  Human CEA with introduction of tetanus toxoid P2 and P30 epitopes
gctagccaccatggaaagtccctcagccccaccccaccgctggtgtattccttggcagcgcctgctcctgacagcaagcctgctgaccttttggaatccacccacgacagccaaactgactatcgagtctactcctttcaacgtggcggaagggaaggaggttctgctcctggtccataatctgccacagcacctgtttggttattcatggtacaaaggcgaacgcgtggatggaaaccggcaaattatcgggtatgttatcggcacacagcaggccacacccggtccggcttacagtgggcgcgaaattatctatccaaatgccagcctgctgatccaaaacattatccagaatgacactggcttctacacactgcatgtcatcaaaagcgatctggtgaacgaggaagcaacgggtcagtttcgggtttatcccgaactgcctaagccatcaatttcttccaataacagtaaacccgtcgaagacaaagacgccgtggctttcacgtgcgagcctgaaacacaagatgcaacttacctgtggtgggttaacaatcagagcctgccagtctcaccccgcctgcagctgagcaacggaaatcggaccctgacactctttaacgtgacgcgcaatgacactgcctcttataagtgtgagacacaaaaccctgtttcagcgcggcgcagcgatagtgttatcctgaatgtgctgtacggcccagacgctcccaccatttccccgctgaacacttcttatcgctcaggggaaaatctgaacctgagctgccacgccgcaagcaatccacccgcgcagtacagttggttcgtcaacggtacatttcagcaatctactcaggaactgttcatccctaatattacggttaacaattcaggcagctatacatgtcaggctcataactccgacacagggctgaatcggactacagtgacgacgatcacagtctacgccgagccacccaaaccctttattacttctaacaatagtaacccagttgaagatgaggacgcagtggctctgacctgcgaacctgagatccaaaatacaacctatctgtggtgggtcaacaatcagtcactgccagttagcccccgcctgcagctcagcaacgacaatcggactctgacgctgctctctgtgacacgcaacgatgtcggtccatacgaatgtggcattcaaaatgaactgtcagttgaccacagtgatcccgtgatcctgaacgtcctgtatggaccagacgaccctaccattagcccatcctacacttattaccggcccggggttaatctgtctctgagctgccatgccgcgtcaaacccacccgcacagtacagctggctgatcgatggcaatattcagcaacacacacaggagctgttcatcagtaacattacagaaaaaaattctggactgtatacgtgtcaggctaacaattccgccagcggtcatagccgcacaactgtgaagacgatcactgtctctgccgagctgccgaaaccaagtatttcatccaacaatagcaagcccgttgaagacaaagatgcagtggcctttacatgcgagccagaagctcaaaacactacgtacctgtggtgggtcaatggccagtctctgcccgttagcccacggctccagctgagtaacgggaatcgcacactgaccctgttcaacgtgacccggaatgacgcgcgcgcctatgtctgtggaatccaaaactccgttagcgcaaatcggtctgaccctgtgacactggatgtcctgtacggcccagacactcccattatctcaccacccgatagcagttatctgagcggtgctaacctgaatctctcttgccactccgcctcaaacccttctccacagtacagctggcgcatcaatgggattccccagcaacatacgcaggttctgtttatcgcaaaaattacaccaaacaataacggcacctatgcgtgtttcgtgagtaacctggccactggacggaataacagcatcgtcaagtccattacagtttctgcaagccagtacatcaaagctaactccaaattcatcggcatcaccgaactgttcaacaacttcaccgtttccttctggctgcgcgttccaaaagtttccgcttcccacctggaaggtacgtcaccctaatga

SEQ ID NO: 6 - protein, 717 aa, Artificial sequence
  Human CEA with introduction of tetanus toxoid P2 and P30 epitopes
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASQYIKANSKFIGITELFNNFTVSFWLRVPKVSASHLEGTSP

SEQ ID NO: 7 - DNA, 2140 bp, Artificial sequence
  Human CEA with introduction of tetanus toxoid P2 and P30 epitopes
gctagccaccatggaaagtccctcagccccaccccaccgctggtgtattccttggcagcgcctgctcctgacagcaagcctgctgaccttttggaatccacccacgacagccaaactgactatcgagtctactcctttcaacgtggcggaagggaaggaggttctgctcctggtccataatctgccacagcacctgtttggttattcatggtacaaaggcgaacgcgtggatggaaaccggcaaattatcgggtatgttatcggcacacagcaggccacacccggtccggcttacagtgggcgcgaaattatctatccaaatgccagcctgctgatccaaaacattatccagaatgacactggcttctacacactgcatgtcatcaaaagcgatctggtgaacgaggaagcaacgggtcagtttcgggtttatcccgaactgcctaagccatcaatttcttccaataacagtaaacccgtcgaagacaaagacgccgtggctttcacgtgcgagcctgaaacacaagatgcaacttacctgtggtgggttaacaatcagagcctgccagtctcaccccgcctgcagctgagcaacggaaatcggaccctgacactctttaacgtgacgcgcaatgacactgcctcttataagtgtgagacacaaaaccctgtttcagcgcggcgcagcgatagtgttatcctgaatgtgctgtacggcccagacgctcccaccatttccccgctgaacacttcttatcgctcaggggaaaatctgaacctgagctgccacgccgcaagcaatccacccgcgcagtacagttggttcgtcaacggtacatttcagcaatctactcaggaactgttcatccctaatattacggttaacaattcaggcagctatacatgtcaggctcataactccgacacagggctgaatcggactacagtgacgacgatcacagtctacgccgagccacccaaaccctttattacttctaacaatagtaacccagttgaagatgaggacgcagtggctctgacctgcgaacctgagatccaaaatacaacctatctgtggtgggtcaacaatcagtcactgccagttagcccccgcctgcagctcagcaacgacaatcggactctgacgctgctctctgtgacacgcaacgatgtcggtccatacgaatgtggcattcaaaatgaactgtcagttgaccacagtgatcccgtgatcctgaacgtcctgtatggaccagacgaccctaccattagcccatcctacacttattaccggcccggggttaatctgtctctgagctgccatgccgcgtcaaacccacccgcacagtacagctggctgatcgatggcaatattcagcaacacacacaggagctgttcatcagtaacattacagaaaaaaattctggactgtatacgtgtcaggctaacaattccgccagcggtcatagccgcacaactgtgaagacgatcactgtctctgccgagctgccgaaaccaagtatttcatccaacaatagcaagcccgttgaagacaaagatgcagtggcctttacatgcgagccagaagctcaaaacactacgtacctgtggtgggtcaatcagtacatcaaagctaactccaaattcatcggcatcaccgaactgctgcccgttagcccacggctccagctgagtaacgggaatcgcacactgaccctgttcaacgtgacccggaatgacgcgcgcgcctatgtctgtggaatccaaaactccgttagcgcaaatcggtctgaccctgtgacactggatgtcctgtacggcccagacactcccattatctcaccacccgatagcagttatctgagcggtgctaacctgaatctctcttgccactccgcctcaaacccttctccacagtacagctggcgcatcttcaacaacttcaccgtttccttctggctgcgcgttccaaaagtttccgcttcccacctggaaattccccagcaacatacgcaggttctgtttatcgcaaaaattacaccaaacaataacggcacctatgcgtgtttcgtgagtaacctggccactggacggaataacagcatcgtcaagtccattacagtttctgcaagctaatga

SEQ ID NO: 8 - protein, 708 aa, Artificial sequence
  Human CEA with introduction of tetanus toxoid P2 and P30 epitopes
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNQYIKANSKFIGITELLPVSPRLQLSNGNRTLTLFNVTRNDARAYVCGIQNSVSANRSDPVTLDVLYGPDTPIISPPDSSYLSGANLNLSCHSASNPSPQYSWRIFNNFTVSFWLRVPKVSASHLEIPQQHTQVLFIAKITPNNNGTYACFVSNLATGRNNSIVKSITVSAS

SEQ ID NO: 9 - DNA, 2155 bp, Artificial sequence
  Human CEA with introduction of tetanus toxoid P2 and P30 epitopes
gctagccaccatggaaagtccctcagccccaccccaccgctggtgtattccttggcagcgcctgctcctgacagcaagcctgctgaccttttggaatccacccacgacagccaaactgactatcgagtctactcctttcaacgtggcggaagggaaggaggttctgctcctggtccataatctgccacagcacctgtttggttattcatggtacaaaggcgaacgcgtggatggaaaccggcaaattatcgggtatgttatcggcacacagcaggccacacccggtccggcttacagtgggcgcgaaattatctatccaaatgccagcctgctgatccaaaacattatccagaatgacactggcttctacacactgcatgtcatcaaaagcgatctggtgaacgaggaagcaacgggtcagtttcgggtttatcccgaactgcctaagccatcaatttcttccaataacagtaaacccgtcgaagacaaagacgccgtggctttcacgtgcgagcctgaaacacaagatgcaacttacctgtggtgggttaacaatcagagcctgccagtctcaccccgcctgcagctgagcaacggaaatcggaccctgacactctttaacgtgacgcgcaatgacactgcctcttataagtgtgagacacaaaaccctgtttcagcgcggcgcagcgatagtgttatcctgaatgtgctgtacggcccagacgctcccaccatttccccgctgaacacttcttatcgctcaggggaaaatctgaacctgagctgccacgccgcaagcaatccacccgcgcagtacagttggttcgtcaacggtacatttcagcaatctactcaggaactgttcatccctaatattacggttaacaattcaggcagctatacatgtcaggctcataactccgacacagggctgaatcggactacagtgacgacgatcacagtctacgccgagccacccaaaccctttattacttctaacaatagtaacccagttgaagatgaggacgcagtggctctgacctgcgaacctgagatccaaaatacaacctatctgtggtgggtcaacaatcagtcactgccagttagcccccgcctgcagctcagcaacgacaatcggactctgacgctgctctctgtgacacgcaacgatgtcggtccatacgaatgtggcattcaaaatgaactgtcagttgaccacagtgatcccgtgatcctgaacgtcctgtatggacagtacatcaaagctaactccaaattcatcggcatcaccgaactgccagacgaccctaccattagcccatcctacacttattaccggcccggggttaatctgtctctgagctgccatgccgcgtcaaacccacccgcacagtacagctggctgatcgatggcaatattcagcaacacacacaggagctgttcatcagtaacattacagaaaaaaattctggactgtatacgtgtcaggctaacaattccgccagcggtcatagccgcacaactgtgaagacgatcactgtctctttcaacaacttcaccgtttccttctggctgcgcgttccaaaagtttccgcttcccacctggaagccgagctgccgaaaccaagtatttcatccaacaatagcaagcccgttgaagacaaagatgcagtggcctttacatgcgagccagaagctcaaaacactacgtacctgtggtgggtcaatggccagtctctgcccgttagcccacggctccagctgagtaacgggaatcgcacactgaccctgttcaacgtgacccggaatgacgcgcgcgcctatgtctgtggaatccaaaactccgttagcgcaaatcggtctgaccctgtgacactggatgtcctgtacggcccagacactcccattatctcaccacccgatagcagttatctgagcggtgctaacctgaatctctcttgccactccgcctcaaacccttctccacagtacagctggcgcatcaatgggattccccagcaacatacgcaggttctgtttatcgcaaaaattacaccaaacaataacggcacctatgcgtgtttcgtgagtaacctggccactggacggaataacagcatcgtcaagtccattacagtttctgcaagctaatga

SEQ ID NO: 10 - protein, 713 aa, Artificial sequence
  Human CEA with introduction of tetanus toxoid P2 and P30 epitopes
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGQYIKANSKFIGITELPDDPTISPSYTYYRPGVNLSLSCHAASNPPAQYSWLIDGNIQQHTQELFISNITEKNSGLYTCQANNSASGHSRTTVKTITVSFNNFTVSFWLRVPKVSASHLEAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSAS

SEQ ID NO: 11 - DNA, 1627 bp, Artificial sequence
  Truncated human CEA with introduction of tetanus toxoid P2 and P30 epitopes
gctagccaccatggaaagtccctcagccccaccccaccgctggtgtattccttggcagcgcctgctcctgacagcaagcctgctgaccttttggaatccacccacgacagccaaactgactatcgagtctactcctttcaacgtggcggaagggaaggaggttctgctcctggtccataatctgccacagcacctgtttggttattcatggtacaaaggcgaacgcgtggatggaaaccggcaaattatcgggtatgttatcggcacacagcaggccacacccggtccggcttacagtgggcgcgaaattatctatccaaatgccagcctgctgatccaaaacattatccagaatgacactggcttctacacactgcatgtcatcaaaagcgatctggtgaacgaggaagcaacgggtcagtttcgggtttatcccgaactgcctaagccatcaatttcttccaataacagtaaacccgtcgaagacaaagacgccgtggctttcacgtgcgagcctgaaacacaagatgcaacttacctgtggtgggttaacaatcagagcctgccagtctcaccccgcctgcagctgagcaacggaaatcggaccctgacactctttaacgtgacgcgcaatgacactgcctcttataagtgtgagacacaaaaccctgtttcagcgcggcgcagcgatagtgttatcctgaatgtgctgtacggcccagacgctcccaccatttccccgctgaacacttcttatcgctcaggggaaaatctgaacctgagctgccacgccgcaagcaatccacccgcgcagtacagttggttcgtcaacggtacatttcagcaatctactcaggaactgttcatccctaatattacggttaacaattcaggcagctatacatgtcaggctcataactccgacacagggctgaatcggactacagtgacgacgatcacagtctacgccgagccacccaaaccctttattacttctaacaatagtaacccagttgaagatgaggacgcagtggctctgacctgcgaacctgagatccaaaatacaacctatctgtggtgggtcaacaatcagtcactgccagttagcccccgcctgcagctcagcaacgacaatcggactctgacgctgctctctgtgacacgcaacgatgtcggtccatacgaatgtggcattcaaaatgaactgtcagttgaccacagtgatcccgtgatcctgaacgtcctgtatggacagtacatcaaagctaactccaaattcatcggcatcaccgaactgccagacgaccctaccattagcccatcctacacttattaccggcccggggttaatctgtctctgagctgccatgccgcgtcaaacccacccgcacagtacagctggctgatcgatggcaatattcagcaacacacacaggagctgttcatcagtaacattacagaaaaaaattctggactgtatacgtgtcaggctaacaattccgccagcggtcatagccgcacaactgtgaagacgatcactgtctctttcaacaacttcaccgtttccttctggctgcgcgttccaaaagtttccgcttcccacctggaaggtacgtcaccctaatga

SEQ ID NO: 12 - protein, 537 aa, Artificial sequence
  Truncated human CEA with introduction of tetanus toxoid P2 and P30 epitopes
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGQYIKANSKFIGITELPDDPTISPSYTYYRPGVNLSLSCHAASNPPAQYSWLIDGNIQQHTQELFISNITEKNSGLYTCQANNSASGHSRTTVKTITVSFNNFTVSFWLRVPKVSASHLEGTSP

SEQ ID NO: 13 - protein, 15 aa, Clostridium tetani
QYIKANSKFIGITEL

SEQ ID NO: 14 - protein, 21 aa, Clostridium tetani
FNNFTVSFWLRVPKVSASHLE

SEQ ID NO: 15 - protein, 13 aa, Artificial sequence
  Artificial universal MHC Class II DR binding sequence
AKFVAAWTLKAAA

(19) United States
(12) Patent Application Publication
(10) Pub. No.: US 2005/0063952 A1
(43) Pub. Date: Mar. 24, 2005

(54) Immunogenic CEA

(76) Inventors: Steen Klysner, Horsholm (DK); Bjorn Voldborg, Horsholm (DK)

Correspondence Address: FROMMER LAWRENCE & HAUG, 745 FIFTH AVENUE- 10TH FL., NEW YORK, NY, 10151, US

(21) Appl. No.: 10/893,018

(22) Filed: Jul. 16, 2004

Related U.S. Application Data
(63) Continuation-in-part of application No. PCT/DK03/00031, filed on Jan. 17, 2003.
(60) Provisional application No. 60/350,047, filed on Jan. 17, 2002.

(30) Foreign Application Priority Data
Jan. 17, 2002 (DK) PA 2002 00082

(51) Int. Cl. A61K 48/00; C12N 15/86

(52) U.S. Cl. 424/93.2; 435/456

(57) ABSTRACT

The present invention provides for methods for immunizing actively against autologous carcinoembryonic antigen (CEA). The method encompasses that the immune system is engaged with variant CEA which is either administered as a protein vaccine, or is effected expressed by nucleic acid vaccination or live-viral vaccination. Preferred embodiments include immunization with variants that include at least one foreign T-helper epitope introduced in the CEA sequence. Also disclosed is variant proteins, DNA, vectors, and host cells useful for practising the method of the invention.

### FIELD OF THE INVENTION

The present invention relates to therapeutic vaccination (“active therapeutic immunotherapy”) against diseases characterized by cells that express carcinoembryonic antigen, CEA. In particular, the present invention relates to the field of cancer therapy and cancer amelioration, where the cancer is characterized by cells that express CEA.

### BACKGROUND OF THE INVENTION

Vaccination has become standard procedure for the prevention of numerous infectious diseases. The application of vaccines to other diseases, such as cancer, is now possible owing to advances in molecular engineering and a better understanding of tumour immunology. The concept of vaccines for cancer treatment is not new and was suggested nearly 100 years ago. Over the years, many attempts have been made to generate effective cancer “vaccines” from mixtures of tumour cells and infectious particles (so-called Coley's toxins) without much success. During this time, studies of transplantable tumours in animals established the feasibility of tumour rejection through immune-mediated mechanisms. These studies suggested that tumour cells expressed unique antigens (i.e. antigens that were not found on normal cells). These antigens, under appropriate conditions, could be recognised by components of the immune system. Further research identified many of the antigens that induced tumour rejection as normal self-proteins. There are many reasons why self-proteins might be recognised by the immune system, including the presence of mutations in the coding regions of the protein, unusually high expression levels of the protein, and abnormal glycosylation of the protein. The awareness that T lymphocytes (T cells) are significant mediators of tumour rejection has focused attention on the isolation of antigens that are specifically recognised by T cells. T cells recognise antigens as smaller fragments of proteins, so-called T cell epitopes, only after their intracellular degradation and presentation on the cell surface, where they are bound to the major histocompatibility complex. The first T-cell-specific tumour antigen was derived from malignant melanoma cells; subsequently, many other tumour antigens in a variety of tumours have been found to possess T-cell-specific epitopes.

CEA was one of the first tumour-associated antigens to be identified and has been well characterised. CEA is an oncofetal glycoprotein, which is found at high levels in the fetal colon and at lower levels in the normal adult colonic epithelium. CEA occurs at abnormally high levels in several benign disorders and in some malignant tumours, including those of the stomach, small intestine, colon, rectum, pancreas, liver, breast, ovary, cervix and lung. Recently, several T-cell epitopes within CEA that are recognised by human T cells have been described. Several different strategies are now using vaccination to target CEA, and clinical trials have started to yield interesting findings.

Successful vaccination against CEA could affect many individuals who have cancer and even more individuals who are at risk of developing cancer. Thus far, CEA appears to be a promising antigen for vaccine therapy.

Biology of CEA: CEA is a 180-kD glycoprotein that is present at high levels in colon epithelial cells during embryonic development. Levels of CEA are significantly lower in colon tissue of adults, but can become elevated when inflammation or tumours arise in any endodermal tissue, including that in the gastrointestinal tract, respiratory tract, pancreas and breast. CEA was originally isolated from a colon carcinoma specimen in 1965. The construction of monoclonal antibodies against CEA has allowed the detection of the overexpression of CEA protein in a variety of adenocarcinomas, including gastric, pancreatic, small intestine, colon, rectal, ovarian, breast, cervical and non-small-cell lung cancers. Currently, ˜500 000 individuals are diagnosed with CEA-producing tumours each year in the USA alone. Epithelial cells in several non-malignant disorders, including diverticulitis, pancreatitis, inflammatory bowel disease, cirrhosis, hepatitis, bronchitis and renal failure and also in individuals who smoke also express CEA. This fact has made it difficult to use serum CEA determination as a sensitive method for cancer screening. However, serum CEA levels have been useful in monitoring individuals for the recurrence of cancer.

In 1986, the gene that encodes human CEA was localised to chromosome 19, and subsequently cloned (Okinawa S. et al. 1987, Biochem. Biophys. Res. Commun. 142, 511-518). In humans, the CEA gene encodes a messenger RNA (mRNA) that is 3100 base pairs long and translates to a protein that has a molecular weight of 70 kD. The additional weight of the protein is provided by an extensive pattern of carbohydrates that are added by glycosylation enzymes, leading to a final weight of 180 kD. The structure of CEA protein includes an N-terminal sequence followed by three disulphide-linked repeats of 178 amino acids, and a hydrophobic c-terminal. This structure is similar to that of the immunoglobulins, and has established CEA as a member of the superfamily of immunoglobulin genes. A unique feature of CEA is that it is linked via lipid into the membrane, through a glycosylphosphatidylinositol moiety, making it distinct from other members of the CEA. Several other antigens are closely related to CEA, including the non-specific cross-reacting antigen (NCA), biliary-specific glycoprotein (BGP), CEA gene family member CGM-6 and pregnancy-specific glycoproteins. Some of these represent separate species, whereas others may be splice variants of CEA. Currently, 29 separate genes have been identified as coding a CEA-related gene product, and most of these genes are located on the long arm of chromosome 19.

The function of CEA in normal colon epithelial cells and in tumour cells is not entirely clear. Many members of the immunoglobulin gene family serve as recognition markers, and this might be true for CEA as well. Studies have reported that CEA localised on the cell surface of colon tumours and other cells can act as a homotypic adhesion molecule, resulting in the aggregation of CEA-expressing cells. Furthermore, although CEA is produced at low levels in normal colonic epithelial cells in adults, the pattern of localisation differs from that observed in most colon tumour cells or in the developing embryonic colon. In normal colonocytes, CEA is localised only at the luminal surface of the cells, whereas in tumour cells, it is found in a disordered pattern throughout the cell membrane. Thus, current models suggest that CEA promotes the spatial orientation of colon epithelial cells to one another and to the surrounding matrix during embryonic development of the colon, and helps maintain the integrity of the luminal epithelium in the adult colon. The altered pattern of localisation in tumour cells may help to disrupt the intercellular adhesion of colonocytes, resulting in the disorganised growth and movement of malignant cells. CEA may also be involved in the enhancement of metastatic disease. Elevated levels of CEA in the serum have been shown to correlate with an increased incidence of liver metastases, and this may be due to adhesion between circulating CEA in the liver and CEA bound to metastatic tumour cells. This may explain the high incidence of hepatic metastases in those patients who have primary tumours that express CEA.

Several different lines of investigation have suggested the possibility that CEA can serve as an antigenic target for eliciting anti-cancer immune responses. Adaptive immune responses to any antigen can be broadly characterised by the production of specific antibodies (i.e. humoral immunity) or the generation of antigen-specific T cells (i.e. cellular immunity). Shortly after the discovery of CEA protein, several groups sought to determine whether individuals who had colon cancer developed anti-CEA antibodies during the course of their disease (Ura Y et al. 1985, Cancer Lett. 25, 283-295; Frenoy N et al. 1987, Anticancer Res 7, 1229-1233). Some groups did not find significantly elevated titres of such antibodies; however, others did find evidence that antibody responses to CEA occurred in some individuals. The potential for CEA to elicit T-cell responses was first suggested by the observation that individuals who had colon cancer often exhibited a delayed-type hypersensitivity (DTH) response to purified CEA protein (Orefice S et al. 1982, Tumori 68, 473-475). More recently, recombinant vaccinia virus expressing CEA were administered to cancer patients, and CEA-specific T cells were subsequently cloned from these patients, demonstrating that T cells can recognise CEA (Tsang K Y et al. 1995, J Natl Cancer Inst 87, 982-990). Several independent groups have now reported the existence of multiple epitopes within CEA that are recognised by human T cells that bind to various HLA class I molecules (Tsang K Y et al. 1995, J Natl Cancer Inst 87, 982-990; Kawashima I et al. 1999, Cancer Res 59, 431-435; Nukaya I et al. 1999, Int J Cancer 80, 92-97).

Prior strategies for CEA cancer vaccine development: Two lines of evidence have supported the use of CEA as a target for vaccine development: the high level of expression of the CEA gene in many different human tumours, and the emerging information about the molecular biology and immunology of CEA. The experimental generation of monoclonal antibodies against CEA paved the way for a variety of diagnostic and therapeutic approaches for cancer therapy based on the detection and targeting of CEA. These have included the direct, in vivo use of anti-CEA monoclonal antibodies, either alone or coupled to radioisotopes or cellular toxins, and also the use of anti-idiotype antibodies. Strategies that target CEA-reactive T cells have also been proposed, including the use of specific HLA-restricted peptides derived from CEA, recombinant viruses and bacteria expressing CEA peptides or proteins, and the pulsing of CEA into antigen-presenting cells (APCs). Dendritic cells are the most potent type of APC. Thus, dendritic cells loaded with CEA peptides, DNA or RNA have been used to stimulate T cells (Nair S K et al. 1999, Int J Cancer 82, 121-124).

Monoclonal-antibody therapy: Monoclonal antibodies directed against CEA were initially used for diagnostic purposes, including the immunohistochemical staining of tissue specimens and the localisation of disease in vivo. The use of antibody-targeted therapeutics for cancer treatment has shown that tumour-cell lysis can be initiated by immune-mediated mechanisms. Antibodies can also be used for the direct delivery of cytotoxic molecules such as radionuclides, toxins or chemotherapy agents to the site of an established tumour.

The discovery that anti-CEA antibodies can be used to detect CEA-expressing tumour cells hinted that they could also be used to mediate the rejection of tumour cells through immune mechanisms. When anti-CEA antibodies bind to the surface of a tumour cell, several pathways are activated, which can result in the destruction of an antibody-marked cell. The presence of bound antibody can activate the complement cascade, leading to cell lysis (complement-mediated cytotoxicity). Another pathway that might be more relevant to tumour cells is the initiation of antibody-directed cellular cytotoxicity (ADCC). This cytotoxic reaction occurs when the Fc portion of an antibody binds to and triggers Fc-receptor-bearing natural killer cells to release cytotoxic granules that lyse cells that are coated with the antibody. However, these anti-tumour effects depend on the presence of CEA on the surface of targeted tumour cells, and because CEA is often found in a heterogeneous pattern, it is difficult to eradicate all of the cells within a tumour mass. Furthermore, the anti-CEA antibody must be able to circulate throughout the body and penetrate solid tumours. This often cannot occur owing to an inadequate blood supply to the tumour. Because many of the monoclonal antibodies that are developed for in vivo clinical use are derived from mice, strong human anti-mouse antibodies (HAMA) can be induced in the patient upon repeated use of the monoclonal antibody; thus, the mouse monoclonal antibody is eliminated before it reaches the tumour.

Using another approach, anti-idiotype antibodies can be used to either elicit or amplify an antigen-specific immune response. For example, immunisation with CEA protein induces the production of Ab1 antibodies. The antigen-binding site of an Ab1 antibody contains a hypervariable complementarity-determining region, which is complementary to the epitope on the antigen that is bound by the antibody. This region is also known as the idiotype, and can induce the production of host antibodies. Immunisation with these idiotypes generates a series of anti-idiotype antibodies, known as Ab2 antibodies, which can resemble some of the epitopes of the original antigen. Thus, Ab2 antibodies then induce the production of anti-anti-idiotype antibodies (Ab3 antibodies), which can specifically bind to the original antigen. The experimental in vivo use of an Ab2 antibody generated against CEA protein in mice has been described; Ab2-immunised mice were protected against challenge with lethal doses of CEA-expressing tumours (Pervin S et al. 1997, Cancer Res 57, 728-734). Clinical trials of this antibody have been conducted, and most patients did develop Ab3 responses that were specific for CEA (Foon K A et al. 1995, J Clin Invest 96, 334-342). Furthermore, one of four patients tested also developed T-cell responses to CEA, although no objective clinical responses were observed (Foon K A et al. 1997, Clin Cancer Res 3, 1267-1276).

Another method of using monoclonal antibodies for cancer therapy is to conjugate them to a radionuclide, which can deliver damaging radiation to the vicinity of the tumour. The advantage of this approach is that targeting a single cell expressing CEA can also lead to the death of nearby tumour cells that are not expressing CEA, owing to a by-stander effect. A similar approach can be used to deliver chemotherapeutic drugs that are known to be toxic to the tumour cells. This is accomplished by conjugating the chemotherapeutic drug to the anti-CEA antibody. The administration of the conjugated antibody results in the accumulation of toxic drug at the site of the tumour rather than in normal tissues. Yet another approach is the construction of genetically modified monoclonal antibodies that are fused with cellular toxins, such as ricin. All of these specialised antibodies can have a by-stander effect, avoiding the problem of heterogeneous CEA expression, but because of the size of the antibody conjugates, delivery and HAMA responses are still problematic. The use of humanised monoclonal antibodies, or chimaeric antibodies that contain only the murine variable regions that interact with antigen combined with human Fc portions, seems to avoid or reduce the HAMA response. However, the delivery of such antibodies to the tumour site still remains a problem. The variable region of the antibody is used to target the cells, and is contained within the Fab portion of the antibody molecule. Because only the Fab fragment is necessary for antigen recognition, smaller antibody fragments containing the Fab protein can be used for targeting tumour cells and enhancing delivery to sites of tumour growth.

CEA-derived peptides: T cells appear to play a major role in tumour rejection after vaccination. Antibodies recognise their antigens by the three-dimensional structure of a single antigenic determinant, the so-called epitope. However, T cells recognise antigen only after the antigen has been processed into smaller linear peptide fragments, which are also known as T cell epitopes. These epitopes are loaded onto specific molecules called major histocompatibility complex (MHC) proteins, so called because they are known to mediate transplantation rejection. MHC class I molecules are found on all nucleated cells, and are recognised by the T-cell receptors (TCRs) of CD8+ T cells. MHC class II molecules are mainly expressed by APCs, and are recognised by CD4+ T cells. As will appear from the present specification and claims, especially peptides binding to MHC Class I molecules would be of interest since these are necessary in order to stimulate a CTL response against a CEA bearing tumour cell.

Several experimental approaches have been used to identify CEA epitopes that are presented by MHC class I molecules to CD8+ T cells. Thus far, the most commonly used approach has been the identification of a putative peptide sequence by using a computer to predict binding affinity to specific MHC class I molecules. This is now easy because the amino acid sequence of the CEA protein has been determined and all nine potential amino acid sequences can be quickly modelled. The peptide groove of an MHC class I molecule normally binds short peptide fragments that comprise 8-10 amino acids, and because the three-dimensional structure of several MHC molecules is known, the computer models can predict the potential peptides that would be expected to bind with high affinity (Rammensee H G et al. 1995, Immunogenetics 41, 178-228). These peptides can be synthesised and tested in vitro for their actual binding affinity for the MHC molecule and for recognition by specific CTLs (Celis E et al. 1994, PNAS USA 91, 2105-2109; Hill A V et al. 1992, Nature 360, 434-439; Houbiers J G et al. 1993, Eur J Immunol 23, 2072-2077).

The above-described method was used to isolate the first HLA-restricted CEA peptide, namely carcinoembryonic-antigen-associated peptide 1 (CAP-1). CAP-1 peptide binds to the HLA-A2 complex, and has been used to generate T-cell lines (i.e. a mixed T-cell population that responds to CEA) from cancer patients who have been vaccinated with recombinant vaccinia virus expressing CEA. A T-cell clone (i.e. a single, genetically identical T-cell population that recognises CEA) derived from one of these patients has been shown to lyse target cells that contain CAP-1 and the HLA-A2 complex. To date, several CEA peptides that specifically bind to known HLA molecules have been identified and have elicited T-cell responses. These peptides can be used to immunise individuals who express the same HLA molecule if these individuals do not tolerate the epitope.

Modified CEA peptides: CEA is a self-antigen and is generally considered to be weakly immunogenic if not non-immunogenic. The main reason for this is that CEA does not include T_{H} epitopes that can induce CD4+ cells to provide the necessary helper functions to B lymphocytes and CTLs.

One method for enhancing recognition is to alter the affinity of a CEA peptide for MHC molecules or T-cell receptors by amino acid substitutions of peptide anchor residues or non-anchor residues, respectively. This strategy can be applied to any known peptide epitope and might increase the immunogenicity of self-antigens. Modifications in the anchor binding residues have resulted in higher affinity binding and better T-cell responses for several melanoma antigens. The CEA peptide CAP-1 was modified by replacing an asparagine residue (N) with an aspartic acid residue (D) at position 6 (Zaremba S et al. 1997, Cancer Res 57, 4570-4577). The resulting “agonist” peptide, designated CAP-1-6D, was recognised by T cells more efficiently than the native CAP-1. Although modified peptides can be used as therapeutic vaccines, the CAP-1-6D peptide has yet to be tested in clinical trials.

Recombinant CEA protein: MHC-class-1-restricted CEA peptides have, as mentioned above, been identified and have been shown to generate CEA-specific T-cell responses; however, such peptides can be used in a clinical setting to treat only those patients whose MHC type is analogous to that of the peptide. Additionally, effective anti-tumour immune responses might depend on the presentation of multiple CEA epitopes through all available MHC molecules expressed in each individual. This increase in peptide diversity can be accomplished by delivering the full-length protein to APCs. Sources of CEA protein include preparations from either tumour biopsy specimens and/or supernatants derived from tumour-cell lines, both of which can contain contaminants. Studies have shown that better humoral and cellular immune responses were elicited in mice by priming (i.e. administering a first vaccination) with recombinant vaccinia virus containing CEA followed by boosting (i.e. administering a second vaccination) with recombinant CEA protein than by vaccination with either virus or protein alone (Bei R et al. 1994, J Immunother Emphasis Tumor Immunol 16, 275-282). Also baculovirus systems have been examined. Intramuscular injection of recombinant baculovirus containing human CEA protein has been evaluated in a clinical trial involving five individuals who had metastatic breast cancer that responded to hormonal therapy. Two of the patients produced both lymphoproliferative responses (i.e. T-cell stimulation and growth) to recombinant CEA protein and strong DTH responses (as revealed by a skin test) after immunisation (Conry R M et al. 1995, J. Immunother. 18, 137). In another clinical trial, patients who had colorectal carcinoma were immunised using either recombinant baculovirus containing human CEA alone or in combination with the cytokine granulocyte-macrophage colony-stimulating factor (GM-CSF). All six of the patients who received the combination treatment showed early CEA-specific T-cell proliferation after immunisation, whereas only two of the six patients who were immunised with recombinant baculovirus containing CEA without GM-CSF developed an anti-CEA T-cell response after multiple vaccinations (Fagerberg J et al. 1995, J. Immunother. 18, 132). These studies provide good evidence for the use of recombinant CEA protein as a boost following primary viral immunisation, or for its use in combination with immune-stimulatory cytokines.

DNA vaccines: DNA vaccines consist of a bacterial plasmid that contains genes (e.g. pathogens, allergens or tumour antigens) that are under the control of a strong eukaryotic promoter. The DNA is usually taken up into host cells, where the encoded antigen is produced and processed via both MHC class I and II pathways, inducing CD8+ and CD4+ T-cell responses. In contrast to viral vaccines, DNA vaccines are relatively simple to produce; moreover, they do not inhibit the immunological responses (e.g. downregulate the MHC class I pathway) that are often associated with viral infections. Naked DNA (i.e. plasmid DNA in saline) has been used for vaccination; this resulted in stable expression after intramuscular injection and the induction of both cellular and humoral (antibody) immune responses. The immune mechanisms involved are only partly understood. It has been suggested that nucleic acid might be taken up selectively by macrophages and/or APCs in the muscle. These activated macrophages then migrate to draining lymph nodes, where they stimulate naive T cells.

A plasmid that encoded the full-length human CEA has been tested by injecting it intramuscularly into mice; both CEA-specific humoral and cell-mediated immune responses were induced. This DNA vaccine also protected mice from a challenge with CEA-expressing colon tumours (Conry R M et al. 1994, Cancer Res 54, 1164-1168).

Dendritic cells: Dendritic cells are the most potent APCs and present antigen via the MHC class I and MHC class II pathways. The use of dendritic cells that have been pulsed (i.e. exposed for a short time to high concentrations) with specific antigens has been proposed as a means of generating more-effective antigen-specific T-cell responses against CEA. In a Phase I study, patients who had advanced malignancies expressing CEA were vaccinated with dendritic cells that had been pulsed with the CEA peptide CAP-1. A minor clinical response was observed for one of the patients in the study, and disease progression was stabilised in another (i.e. there was no tumour growth following vaccination). No treatment-related toxicities were observed, ting the feasibility and safety of this treatment method (Morse M A et al. 1999, Clin Cancer Res 5, 1331-1338). Dendritic cells that had been pulsed with a cocktail of melanoma peptides or a tumour lysate were used to treat patients who had advanced melanoma by injecting the cells into or near lymph nodes. Five patients out of 16 produced a clinical response to the vaccine, and two of the five responded completely (Nestle F O et al. 1998, Nat Med 4, 328-332).

Another approach used CEA-specific mRNA and total RNA derived from CEA-expressing tumour cells. RNA encodes multiple CEA epitopes for various HLA types meaning that patients can be immunised without the need for prior identification of their HLA type or the use of HLA-specific CEA epitope(s). Moreover, RNA can be extracted from very small amounts of tumour tissue and encodes the individual array of tumour antigens for that tumour. Studies that utilised autologous dendritic cells that had been pulsed with either CEA peptides or CEA RNA to stimulate isolated T cells from carcinoma patients and healthy donors showed that a CEA-specific CTL response could be elicited in vitro (Alters S E et al. 1997, Adv Exp Med Biol 417, 519-524; Alters S E et al. 1998, J Immunother 21, 17-26; Nair S K et al. 1999, Int J Cancer 82, 121-124). Another method of generating immunogenic vaccines is to fuse whole tumour cells directly to dendritic cells, using an electrofusion technique. In a pilot study involving patients who had renal cell carcinoma, the administration of a fusion vaccine composed of autologous renal cell carcinoma cells fused to allogeneic dendritic cells produced a significant clinical response in seven of the 17 treated patients, four of which showed complete responses (Kugler A et al. 2000, Nat Med 6, 332-336).

Bacterial vaccines: The delivery of DNA that encodes tumour antigens to APCs can also be accomplished using live attenuated bacteria. The advantages of using bacteria as expression vectors for foreign antigens include improved antigen presentation, because some bacteria are engulfed (taken up) by phagosomes, resulting in the presentation of inserted antigens by both MHC class I and class II pathways. Bacteria also provide the requisite transcriptional and translational machinery for the expression of foreign genes. In bacteria, post-translational glycosylation of encoded proteins might be problematic. However, as most vaccines aim to elicit a T-cell response that is dependent on the MHC class I or class II pathway, the presentation of peptides should not be restricted. Another advantage of bacterial vectors is that they are sensitive to antibiotics and can be more easily controlled after administration to patients. Animal studies have shown the therapeutic effectiveness of using recombinant bacterial vaccines for the treatment of model tumours; both CD4+- and CD8+-antigen-specific T-cell responses were generated (Pan Z K et al. 1995, Nat Med 1, 471-477). Bacteria that are amenable to the expression of tumour antigens include Bacillus Calmette-Guerin (BCG), Salmonella typhimurium and Listeria monocytogenes. Bacterial recombinants that express human tumour antigens have not yet been tested in clinical trials. Although promising, further research is needed to better characterise the effects of bacterial vaccines as agents for cancer therapy.

Recombinant viruses: Perhaps the best-studied vaccine development method involves the use of recombinant viruses. The most characterised viral system is that of the poxviruses, particularly vaccinia virus. Recombinant vaccinia viruses can accept a large insert of foreign DNA, replicate accurately, are easily engineered, allow post-translational modification of foreign proteins (e.g. glycosylation), stimulate strong immune responses, and have been extensively used in the human population to prevent and eradicate smallpox. The methods for constructing recombinant vaccinia viruses have been well described, and several different tumour antigens have now been placed into these viruses, including CEA (Mackett M et al. 1982, PNAS USA 79, 7415-7419; Kaufman H et al. 1991, Int J Cancer 48, 900-907).

In a colon carcinoma model in mice, vaccinia virus expressing CEA was found to be effective in treating established CEA-expressing tumours, and was associated with the development of anti-CEA antibody titres and T-cell responses (Kantor J et al. 1992, J Natl Cancer Inst 84, 1084-1091). Interestingly, the vaccine was most effective in preventing the growth of CEA-bearing tumours in pre-immunised animals. The same vaccine has also been tested for safety and immunogenicity in a non-human primate model; toxicity was found to be minimal and the monkeys produced CEA-specific T-cell responses after vaccination (Kantor J. et al. 1992, Cancer Res 52, 6917-6925).

Several clinical trials using recombinant vaccinia vaccine containing the CEA gene to treat patients who had advanced CEA-expressing tumours have provided evidence that vaccination was safe even when high titres of virus were given; CEA-specific T-cell growth and cytotoxicity was also induced.

To circumvent the neutralising antibody responses against the vaccines that are induced in individuals previously subjected to vaccinia virus vaccination, attention has now focused on the use of attenuated vaccinia viruses and non-replicating poxviruses, such as the avipoxviruses. The attenuated vaccinia strains, such as NYVAC and modified vaccinia virus Ankara (MVA), contain multiple gene deletions, which prevent the virus from replicating in mammalian cells. Although avipoxyiruses, including fowlpox and canarypox (ALVAC) virus, are pathogenic in birds, they are also unable to replicate in mammalian cells. However, they are able to elicit strong T-cell immune responses in both rodent models and humans. These T-cell responses have not been accompanied by the induction of strong neutralising antibodies and have allowed repeated immunisations. An ALVAC virus expressing CEA has shown therapeutic effectiveness in a CEA tumour model in mice, and has been tested in human clinical trials (Long L et al. 1999, Curr. Opin. Mol. Ther. 1, 57-63; Marshall J L et al. 1999, J Clin Oncol 17, 332-337).

The safety of viral vaccines and the ability to generate CEA-specific T-cell responses has led to several novel approaches for improving the clinical effectiveness of the vaccines. This includes the addition of adjuvants, such as cytokines and co-stimulatory molecules, to the treatment regimen, and combining different viruses in a ‘prime and boost’ strategy.

Attempts to enhance known CEA cancer vaccines: Several approaches for vaccine design have been presented; however, the results from clinical trials have thus far been disappointing. One reason might be the use of vaccines in patients who have advanced disease, because they are less likely to elicit a measurable and protective immune response. Although such individuals may be able to respond to common antigens (e.g. influenza or tetanus), their response may be locally immunosuppressed at the tumour site. Thus, patients who have advanced cancers may be less likely to respond to vaccination against a tumour-associated antigen. Several strategies could be employed to improve the ability of CEA vaccines to induce immune response.

Cytokines: IL-2 was the first cytokine to be shown to induce tumour regression in an animal model and it has been tested against a variety of human cancers, and has been shown to have therapeutic potential when administered intravenously as a single agent for metastatic melanoma and renal cell carcinoma. Because vaccines can induce T-cell responses, it seems logical that IL-2 could be used to amplify the initial response, improving the therapeutic effects of cancer vaccines. This has been confirmed experimentally in a mouse model, whereby IL-2 significantly augmented the anti-tumour responses of a vaccinia virus expressing CEA (McLaughlin J P et al. (1996, Cancer Res 56, 2361-2367).

Recombinant viral vaccines that encode both tumour antigen and cytokine genes have been constructed. These have been designed to induce the local release of cytokine at the site of T-cell activation, and should limit the systemic toxicity usually induced by the intravenous administration of high doses of IL-2.

The combination of numerous other cytokines with antigen-specific vaccines has improved the effects of tumour treatment methods. IL-12 is a cytokine that is involved in the stimulation of natural killer cells and the differentiation of naive T cells. Thus, IL-12 can be considered as an important mediator of the effector phase of cellular immunity. Other cytokines that have been evaluated for their ability to augment tumour vaccines include GM-CSF, IFN-γ, tumour necrosis factor α (TNF-α), interleukin 3 (IL-3), IL-4 and interleukin 10 (IL-10). GM-CSF has been shown to promote the growth and activation of dendritic cells, thus improving the antigen presentation ‘arm’ of the immune system. Irradiated tumour cells that were transduced ex vivo with the GM-CSF gene have been used as an autologous cellular vaccine and increased the anti-tumour T-cell response.

Co-stimulation of tumour-antigen-specific T cells: The activation of antigen-specific T cells, leading to cytokine production and proliferation, requires two separate signals. The first signal is delivered to the T-cell receptor upon recognition of the peptide-MHC complex. The second signal can be delivered by CD28 molecules expressed on T cells after the engagement of the B7 co-stimulatory molecule expressed by activated APCs. The importance of co-stimulation has been demonstrated by experiments that show that T cells do not respond when peptide-MHC or TCR recognition takes place in the absence of co-stimulatory molecules. Other studies have shown that tumour cells can escape detection and subsequent elimination by T cells by the downregulation of co-stimulatory molecules on the tumour cell surface, or on dendritic cells presenting the tumour antigens.

The B7 co-stimulatory molecules are homodimeric (i.e. express two identical, intertwined chains of the same protein) members of the immunoglobulin supergene family; they are found on the surface of cells that are capable of stimulating T-cell activation and proliferation. B7 molecules can bind to either CD28 or CTLA-4 on the surface of T cells. In the first instance, the ligation of CD28 (e.g. via B7 molecules or anti-CD28 monoclonal antibody) delivers an activating signal to the T cell, which induces the release of cytokines. After activation, T cells upregulate the expression of CTLA-4 on their cell surface, which also binds B7 molecules but delivers a negative signal, rendering the T cells less sensitive to further stimulation. The fate of T cells that respond to an antigenic stimulus depends on the balance between the stimulatory and inhibitory signals delivered to the T cell via these surface receptors. Likewise, T-cell activation can be enhanced by selectively stimulating CD28 or blocking CTLA-4 activity, and it can be inhibited by the reverse treatments.

Using a mouse model, a mixture of vaccinia viruses expressing CEA and B7 molecules resulted in enhanced CEA-specific CTL responses and more-effective anti-tumour activity (Hodge J W et al. 1995, Cancer Res 55, 3598-3603). This and other findings support the use of B7 molecules as a vaccine adjuvant, and suggest that this approach will be safe and might be expected to elicit more-objective clinical responses in larger clinical trials involving patients whose disease is at an earlier stage.

Interactions between CD40 and CD40 ligand (also known as CD40L or CD154) represent another co-stimulatory system that has been widely studied. The CD40 receptor is a 48-kDa protein, which is found on many cell types, especially APCs, such as B cells, dendritic cells, macrophages, monocytes, fibroblasts and endothelial cells. CD40L is a 39-kDa protein that belongs to the TNF family and is predominantly expressed on activated CD4+ T cells. Interactions between CD40 and CD40L are important for priming CTLS by CD4+ T cells, and might also help induce humoral immunity. To date, the potential benefits of increasing the expression of CD40L together with that of CEA to produce a novel tumour vaccine (i.e. vaccinia virus encoding CD40L and CEA) have not been experimentally evaluated.

Prime and boost strategies: The generation of multiple vectors for vaccination, and the development of neutralising antibodies that prevent repetitive exposure to a single vector, has led to the use of prime and boost strategies. Such protocols prime the immune response with one vector expressing an antigen, and then boost with a different vector expressing the same antigen. Heterologous boosting of mice using first vaccinia virus and then ALVAC virus expressing CEA improved tumour responses and CTL activity against CEA, compared to those produced by vaccination with either virus alone (Hodge J W et al. 1997, Vaccine 15, 759-768) and clinical trials are beginning to suggest similar results (Cole D J et al. 1996, Hum Gene Ther 7, 1381-1394).

Induction of T-cell help-mechanisms: Presentation of antigens has dogmatically been thought of as 2 discrete pathways, a class II exogenous and a class I endogenous pathway.

Briefly, a foreign protein from outside the cell or from the cell membrane is taken up by the APC as an endosome that fuses with an intracellular compartment containing proteolytic enzymes and MHC class II molecules. Some of the produced peptides bind to class II, which then are translocated to the cell membrane.

The class I endogenous pathway is characterised by the predominant presentation of cytosolic proteins. This is believed to occur by proteasome-mediated cleavage followed by transportation of the peptides into the endoplasmic reticulum (ER) via TAP molecules located in the membrane of the ER. In ER the peptides bind to class I followed by transportation to the plasma membrane.

However, these 2 pathways are not fully distinct. For example it is known that dendritic cells and to some extend macrophages are capable of endocytosing (pinocytosing) extracellular proteins and subsequently present them in the context of MHC class I. It has also previously been demonstrated that using specialised administration routes, e.g. by coupling to iron oxide beads, exogenous antigens are capable of entering the Class I pathway. This mechanism seems central, because of the importance of a concomitant expression of both class I and class II on the same APC to elicit a three cell type cluster. This three-cell type cluster of interaction has been proposed by Mitchison (1987) and later by other authors. They showed the importance of concomitant presentation of class I and class II epitopes on the same APC. According to the recently described mechanism for CTL activation (cf. Lanzavecchia, 1998, Nature 393: 413, Matzinger, 1999, Nature Med. 5: 616, Ridge et al., 1998, Nature 393: 474, Bennett et al., 1998, Nature 393: 478, Schoenberger et al., 1998, Nature 393: 480, Ossendrop et al., 1998, J. Exp. Med 187: 693, and Mackey et al., 1998, J. Immunol 161: 2094), professional APCs presenting antigen on MHC class II are recognized by T helper cells. This results in an activation of the APC (mediated by interaction by CD40L on the T helper cell and CD40 on the APC). This enables the APC to directly stimulate CTLs that are thereby activated. Cf. also FIG. 2 in WO 00/20027.

It has previously been demonstrated that insertion of a foreign MHC class II restricted T helper cell epitope into a self-antigen results in the provision of an antigen capable of inducing strong cross-reactive antibody responses directed against the non-modified self-antigen (cf. WO 95/05849). It was shown that the autoantibody induction is caused by specific T cell help induced by the inserted foreign epitope.

Later, it was concluded that modified self-antigens—with the aid of appropriate adjuvants—ought to be capable of also inducing strong CTL responses against MHC class I restricted self-epitopes and hence the technology described in WO 95/05849 can be adapted to also provide vaccination against intracellular and other cell-associated antigens which have epitopes presented in the context of MHC Class I—this concept is the subject matter of WO 00/20027 which is hereby incorporated by reference herein.

WO 00/20027 mentions CEA as one putative vaccine target but provides no specific information concerning optimal localisation of foreign T_{H} epitopes within the CEA amino acid sequence.

As will appear from the above discussion of CEA, this particular polypeptide antigen appears to be a safe target for active immunotherapy. However, there is still a definite need to improve the technology for inducing an immune response against this particular antigen.

### OBJECT OF THE INVENTION

It is an object of the present invention to provide improved methods and agents for inducing immune responses in host organisms against cells, especially malignant cells that harbour CEA. It is a further object to provide a method for preparing polypeptide analogues of CEA, analogues that are capable of inducing an effective immune response against CEA and cells that harbour CEA.

### SUMMARY OF THE INVENTION

The inventors have in part based the present invention on the teachings of WO 00/20027 and WO 95/05849 that teach generally applicable methods for providing CTL and antibody responses, respectively, against autologous proteinaceous antigens.

Using the technology disclosed in WO 00/20027, the modified CEA could be presented by MHC class I as well as by MHC class II molecules on professional antigen presenting cells. Co-presentation of subdominant self-epitopes on MHC class I and immunodominant foreign epitopes on MHC class II molecules will mediate a direct cytokine help from activated MHC class II restricted T-helper cells to MHC class I restricted CTLs (FIG. 2 in WO 00/20027). Further, presentation by B-cells of foreign T_{H} epitopes present in variants of CEA will result in induction of production of anti-CEA antibodies, cf. FIG. 1 in WO 00/20027. Hence both strategies as well as their combination will lead to a specific break of the T cell autotolerance towards CEA, thus rendering possible both CTL as well as antibody induction that will specifically target CEA.

The present invention is therefore based on a careful study of the structure of native CEA in order to facilitate the designing of variants of CEA that will prove to be most efficient in breaking tolerance to this antigen. The thus obtained CEA variants are the sine qua non constituents in therapy against CEA expressing cancers, but of course all technologies discussed above in the “Background of the Invention” section can be advantageously combined with the present invention. Therefore, the technologies discussed in said section are all incorporated by reference herein.

In conclusion, a vaccine constructed using both of the technologies outlined above will induce a humeral autoantibody response with secondary activation of complement and antibody dependent cellular cytotoxicity (ADCC) activity. Equally important, it will also induce a cytotoxic T cell response directed against autologous CEA producing cells.

Hence, in the broadest and most general scope, the present invention relates to a method for inducing an immune response against autologous carcinoembryonic antigen (CEA) in an animal, including a human being, the method comprising effecting uptake and processing by antigen presenting cells (APCs) in the animal of at least one modified CEA polypeptide or of a nucleic acid encoding the modified CEA polypeptide or of a pharmaceutically acceptable microorganism or virus expressing the modified CEA polypeptide, said at least one modified CEA polypeptide comprising
at least about 80 CEA-derived amino acids, either in the form of at least about 80 consecutive CEA-derived amino acids or in the form of at least about 80 amino acids constituted of uninterrupted CEA-derived CTL epitopes, and
at least one first T helper epitope (T_{H} epitope) foreign to the animal,
   thereby inducing a CTL response and/or an antibody response that targets the autologous CEA.

Furthermore, the invention relates to certain specific immunogenic constructs based on human CEA as well as to compositions containing these constructs.

Finally, the invention relates to nucleic acid fragments, vectors, transformed cells and other tools useful in molecular biological methods for the production of the analogues of CEA.

### LEGENDS TO THE FIGURE

FIG. 1: Overview of the design strategy for the modified CEA polypeptides of the present invention.

The panel to the left shows a model of CEA with domains 1-7 numbered. The N-terminal of the molecule is at the top, the C-terminus at the bottom. Circles indicate introduction points for T_{H} epitopes in the loops of the 7 domains. Arrows indicate introduction points in the “linkers” between the domains. Triangles show the C- and N-terminal introduction points. The column with heading “struct. design” indicates the optimal introduction regions/points from a strict structural viewpoint, whereas the column headed “MHC Class I corr.” indicates the chosen introduction regions/points after an analysis of possible interference with CTL epitopes of CEA. Asterisks indicate that the introduction region includes a glycosylation point.

FIG. 2: Schematic overview of 4 preferred CEA variants. The panel to the left shows a schematic view of the CEA domains and the 4 models schematically show the variants having the amino acid sequences set forth in SEQ ID NOs: 6, 8, 10, and 12, respectively.

### DETAILED DISCLOSURE OF THE INVENTION

Definitions

In the following a number of terms used in the present specification and claims will be defined and explained in detail in order to clarify the metes and bounds of the invention.

An “autologous CEA” is in the present specification and claims intended to denote a CEA polypeptide of an animal that is going to be vaccinated against its own CEA. In other words, the term is only relevant when the relation to the animal that it going to be vaccinated is considered.

The terms “T-lymphocyte” and “T-cell” will be used interchangeably for lymphocytes of thymic origin which are responsible for various cell mediated immune responses as well as for effector functions such as helper activity in the humeral immune response. Likewise, the terms “B-lymphocyte” and “B-cell” will be used interchangeably for antibody-producing lymphocytes.

An “antigen presenting cell” (APC) is a cell which presents epitopes to T-cells. Typical antigen-presenting cells are macrophages, dendritic cells and other phagocytizing and pinocytizing cells. It should be noted that B-cells also functions as APCs by presenting T_{H} epitopes bound to MCH class II molecules to T_{H} cells but when generally using the term APC in the present specification and claims it is intended to refer to the above-mentioned phagocytizing and pinocytizing cells.

“Helper T-lymphocytes” or “T_{H} cells” denotes CD4 positive T-cells which provide help to B-cells and cytotoxic T-cells via the recognition of T_{H} epitopes bound to MHC Class II molecules on antigen presenting cells.

The term “cytotoxic T-lymphocyte” (CTL) will be used for CD8 positive T-cells which require the assistance of T_{H} cells in order to become activated.

A “specific” immune response is in the present context intended to denote a polyclonal immune response directed predominantly against a molecule or a group of quasi-identical molecules or, alternatively, against cells which present CTL epitopes of the molecule or the group of quasi-identical molecules.

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring amino acid sequence or nucleic acid sequence, respectively.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention all harbour substantially the same CEA allowing for immunization of the animals with the same immunogen(s). If, for instance, genetic variants of CEA exist in different human populations it may be necessary to use different immunogens in these different populations in order to be able to break the autotolerance towards the CEA in each population.

By the term “down-regulation a autologous CEA” is herein meant reduction in the living organism of the amount and/or activity of CEA. The down-regulation can be obtained by means of several mechanisms including removal of the CEA by scavenger cells (such as macrophages and other phagocytizing cells), and even more important, that cells carrying or harbouring the antigen are killed by CTLs in the animal.

The expression “ . . . consecutive CEA-derived amino acids . . . ” is intended to denote amino acids that can be found in the same sequential order in the primary structure of a naturally occurring CEA, nevertheless allowing for conservative substitutions that do not change the immunologic properties of such a CEA sequence.

The expression “ . . . amino acids constituted of uninterrupted CEA-derived CTL epitopes . . . ” is meant a stretch of amino acids that can be subdivided into shorter consecutive stretches of CEA that each constitute a CTL epitope found in naturally occurring CEA. Again, the language allows for minor, insignificant changes in the amino acid sequences of these CTL epitopes, as long as the CTL epitopes are not rendered less immunoreactive than their natural form.

The expression “effecting simultaneous presentation by a suitable APC” is intended to denote that the animal's immune system is subjected to an immunogenic challenge in a controlled manner which results in the simultaneous presentation by APCs of the CEA epitopes and foreign epitopes in question. As will appear from the disclosure below, such challenge of the immune system can be effected in a number of ways of which the most important are vaccination with polypeptide containing “pharmaccines” (i.e. a vaccine which is administered to treat or ameliorate ongoing disease) or nucleic acid “pharmaccine” vaccination. The important result to achieve is that immune competent cells in the animal are confronted with APCs displaying the relevant epitopes in an immunologically effective manner.

The term “immunogen” is intended to denote a substance which is capable of inducing an immune response in a certain animal. It will therefore be understood that autologous CEA is not an immunogen in the autologous host—it is necessary to use either a strong adjuvant and/or to co-present T helper epitopes with the autologous CEA in order to mount an immune response against autologous CEA and in such a case the “immunogen” is the composition of matter which is capable of breaking autotolerance.

The term “immunogenically effective amount” has its usual meaning in the art, i.e. an amount of an immunogen which is capable of inducing an immune response which significantly engages pathogenic agents which share immunological features with the immunogen.

The term “pharmaceutically acceptable” has its usual meaning in the art, i.e. it is used for a substance that can be accepted as part of a medicament for human use when treating the disease in question and thus the term effectively excludes the use of highly toxic substances that would worsen rather than improve the treated subject's condition.

When using the expression that the autologous CEA has been subjected to a “modification” is herein meant a chemical modification of the polypeptide which constitutes at least part of one of the 7′ domains of autologous CEA. Such a modification can e.g. be derivatization (e.g. alkylation) of certain amino acid residues in the amino acid sequence, but as will be appreciated from the disclosure below, the preferred modifications comprise changes of the primary structure of the amino acid sequence.

When discussing “tolerance” and “autotolerance” is understood that since CEA molecules which are the targets of the present inventive method are self-proteins in the population to be vaccinated, normal individuals in the population do not mount an immune response against CEA. It cannot be excluded, though, that occasional individuals in an animal population might be able to produce antibodies against the autologous CEA, e.g. as part of a autoimmune disorder. At any rate, an animal will normally only be autotolerant towards its own CEA, but it cannot be excluded that analogues derived from other animal species or from a population having a different phenotype would also be tolerated by said animal.

A “foreign T-cell epitope” is a peptide which is able to bind to an MHC molecule and which stimulates T-cells in an animal species. Preferred foreign epitopes are “promiscuous” epitopes, i.e. epitopes which binds to a substantial fraction of MHC class II molecules in an animal species or population. A term which is often used interchangeably in the art is the term “universal T-cell epitopes” for this kind of epitopes. Only a very limited number of such promiscuous T-cell epitopes are known, and they will be discussed in detail below. It should be noted that in order for the immunogens which are used according to the present invention to be effective in as large a fraction of an animal population as possible, it may be necessary to 1) insert several foreign T-cell epitopes in the same analogue or 2) prepare several analogues wherein each analogue has a different promiscuous epitope inserted. It should be noted that the concept of foreign T-cell epitopes also encompasses use of cryptic T-cell epitopes, i.e. epitopes which are derived from a self-protein and which only exerts immunogenic behaviour when existing in isolated form without being part of the self-protein in question.

A “foreign T helper lymphocyte epitope” (a foreign T_{H} epitope) is a foreign T cell epitope which binds an MHC Class II molecule and can be presented on the surface of an antigen presenting cell (APC) bound to the MHC Class II molecule. It is also important to add that the “foreignness” feature therefore has two aspects: A foreign T_{H} epitope is 1) presented in the MHC Class II context by the animal in question and 2) the foreign epitope is not derived from the same polypeptide as the target antigen for the immunization—the epitope is thus also foreign to the target antigen.

A “CTL epitope” is a peptide which is able to bind to an MHC class I molecule.

A “functional part” of a (bio)molecule is in the present context intended to mean the part of the molecule which is responsible for at least one of the biochemical or physiological effects exerted by the molecule. It is well-known in the art that many enzymes and other effector molecules have an active site which is responsible for the effects exerted by the molecule in question. Other parts of the molecule may serve a stabilizing or solubility enhancing purpose and can therefore be left out if these purposes are not of relevance in the context of a certain embodiment of the present invention. For instance it is possible to use certain cytokines as a modifying moiety in the modified CEA (cf. the detailed discussion below), and in such a case, the issue of stability may be irrelevant since the coupling to the modified CEA provides the stability necessary.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Targeting” of a molecule is in the present context intended to denote the situation where a molecule upon introduction in the animal will appear preferentially in certain tissue(s) or will be preferentially associated with certain cells or cell types. The effect can be accomplished in a number of ways including formulation of the molecule in composition facilitating targeting or by introduction in the molecule of groups which facilitates targeting. These issues will be discussed in detail below.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen

Preferred Embodiments

The present invention targets CEA via Active Specific Immunotherapy—in essence this can be achieved by stimulating either or both of the two arms of the immune system. Since CEA is a membrane bound protein that is exposed mostly to the extracellular phase and thereby open for antibody interaction, it is expedient to raise an antibody response against CEA, i.e. a humoral response. On the other hand, as discussed above, cell-mediated immunity (e.g. a CTL response) against CEA-bearing cells should also be a feasible means of attacking this particular antigen and diseases related thereto.

According to the invention, it is therefore possible to 1) use an immunogen that induces CTLs reactive with CEA as well as antibodies reactive with CEA, 2) use an immunogen that only induces CTLs reactive with CEA, 3) use an immunogen that only induces antibodies reactive with CEA, or use a combination of 2 and 3 (which both can be combined with 1).

From a practical viewpoint, it is interesting to include a large fraction of CEA epitopes. So, even though it is believed to be sufficient to include down to about 80 CEA-derived amino acids, longer parts of CEA are preferred: at least about 100, 120, 140, 160, 180, 200, 220, 250, 300, 350, 400, 450, 500, 550, 600, 650, 700, thus even up to about the full sequence of CEA. However, since the molecule consists of domains (each having a length of about 100 amino acids) it is especially preferred to substantially include the amino acid sequence of at least one domain, such as at least 2, 3, 4, 5, 6 or even all 7 domains of CEA (still allowing for the minor variations discussed above). The domains of CEA are consecutively numbered from the N-terminus of the molecule. To “substantially include” means in this context that the amino acid sequence of such a domain may be subjected to minor variations in the form of conservative substitutions that will not significantly alter the immunologic profile of such a domain. The term also means, that in the event a foreign T_{H} epitope is introduced according to the invention in a domain region, this is done in a manner that will not significantly interfer with the 3-dimensional structure of the domain region (this can e.g. be achieved by introducing the epitope in the loop in one or more of the domains, cf. below. It is important to note that if it is desired to merely induce CTL responses, the requirement of preservation of 3-dimensional structure of one or more domains is irrelevant.

In order to induce a CTL response against a cell which presents epitopes derived from the autologous CEA on its surface, it is normally necessary that at least one CTL epitope, when presented, is associated with an MHC Class I molecule on the surface of the APC. Furthermore it is preferred that the at least one first foreign T_{H} epitope, when presented, is associated with an MHC Class II molecule on the surface of the APC.

Preferred APCs presenting the CTL epitopes are dendritic cells and macrophages, but any pino- or phagocytizing APC which is capable of simultaneously presenting 1) CTL epitopes bound to MHC class I molecules and 2) T_{H} epitopes bound to MHC class II molecules, is a preferred APC according to the invention.

Normally, it will be advantageous to confront the immune system with a large fraction of the amino acid sequence of the autologous CEA—this will ensure that a large number of CTL epitopes and/or B-cell epitopes will be present in the agent that is taken up by the APC. Hence, in a preferred embodiment, the modified CEA polypeptide is in the form of at least one first analogue of the autologous CEA, said first analogue containing autologous CEA-derived CTL epitope(s), and the at least first foreign T_{H} epitope.

In order to maximize the chances of mounting an effective immune response, it is preferred that the above-mentioned first analogue contains a substantial fraction of known and predicted CTL epitopes of autologous CEA, i.e. a fraction of the known and predicted CTL epitopes which binds a sufficient fraction of MHC Class I molecules in a population. For instance, it is preferred that the substantial fraction of known and predicted CTL epitopes in the amino acid sequence of the analogue are recognized by at least 50% of the MHC—I haplotypes recognizing all known and predicted CTL epitopes in the autologous CEA, but higher percentages are preferred, such as at least 60, at least 70, at least 80, and at least 90%. Especially preferred is the use of analogues, which preserve substantially all known CTL epitopes of the autologous CEA are present in the analogue, i.e. close to 100% of the known CTL epitopes. Accordingly, it is also especially preferred that substantially all predicted CTL epitopes of the autologous CEA are present in the at least first analogue.

The above-indicated approach renders possible the mounting of a CTL response against all parts of cell-associated CEA, including the membrane-anchoring region and also regions of CEA that are in close proximity to the cell membrane and therefore may be shielded by other cellular molecules, thereby making these regions less effective as targets for humoral immune responses.

Methods for predicting the presence of CTL epitopes are well-known in the art, cf. e.g. Rothbard et al. EMBO J. 7:93-100 (1988) and Rammensee H G et al. 1995, Immunogenetics 41, 178-228. Furthermore, a number of CTL epitopes have already been identified in CEA.

As will be apparent from the present specification and claims it is expected that the inventive method described herein will render possible the effective induction of CTL responses against autologous CEA.

Since CEA is a membrane-associated antigen, it is according to the present invention also advantageous to induce an antibody response while at the same time inducing CTL mediated immunity, cf above. However, when raising a humeral immune response against autologous CEA it is preferred to substantially restrict the antibody response to interaction with the parts of the antigen that are normally exposed to possible interaction with antibodies (i.e. those regions being relatively remote from the cell membrane). Otherwise the result could possibly be the induction of an antibody response against parts of the antigen which is not normally engaging the humeral immune system, and this will in turn increase the risk of inducing cross-reactivity with antigens not related to any pathology—even though this is not regarded a serious risk, the size of CEA renders truncated forms of the molecule likely candidates for a vaccine. One elegant way of obtaining this restriction is to perform nucleic acid vaccination with an analogue of autologous CEA, where the extracellular part thereof is either unaltered or includes a T_{H} epitope which does not substantially alter the 3D structure of the relevant extracellular part of the antigen. As one possible alternative, immunization can be performed with both a CTL directed immunogen and a B-cell directed immunogen where the B-cell directed immunogen is substantially incapable of effecting immunization against the cell-proximal part part of the target antigen (the B-cell directed immunogen could e.g. include only the first 3 domains of CEA—domains 2, 4 and 6 share a very high homology and the same is true fo for domain 3, 5 and 7 and the inclusion of the first 3 domains therefore ought to provide the desired variability with respect to B-cell epitopes in the immunogen).

Induction of antibody responses can be achieved in a number of ways known to the person skilled in the art. For instance, the at least one first analogue may comprise at least one CEA B-cell epitope, so that immunization of the animal with the first analogue also induces production of antibodies in the animal against the autologous CEA—this type of analogue is as mentioned above very well suited for nucleic acid vaccination. Alternatively, the method of the invention can involve effecting presentation to the animal's immune system of an immunogenically effective amount of at least one second analogue (which shares the generic definition of a “modified CEA polypeptide” used herein). A convenient way to achieve that the second analogue has the desired antibody-inducing effect is to include at least one second foreign T_{H} epitope in the second analogue, i.e. a strategy like the one used for the first analogue. This at least one second T_{H} epitope may be the same or different from the at least one first T_{H} epitope.

In the cases where it is desired to also mount an effective humoral immune response, it is advantageous that the first and/or second analogue(s) comprise(s) a substantial fraction of the B-cell epitopes of autologous CEA, especially a substantial fraction of such B-cell epitopes which are exposed to antibody interaction in the naturally occurring form of autologous, membrane bound CEA, cf. the above discussion of the region including domains 1-3.

The above-discussed variations and modifications of the autologous CEA can take different forms. It is preferred that the variation and/or modification involves amino acid substitution and/or deletion and/or insertion and/or addition. These fundamental operations relating to the manipulation of an amino acid sequence are intended to cover both single-amino acid changes as well as operations involving stretches of amino acids (i.a. shuffling of amino acid stretches within the polypeptide antigen; this is especially interesting when only aiming at inducing CTLs, since only considerations concerning preservation of CTL epitopes are relevant). It will be understood, that the introduction of even as little as one single amino acid insertion or deletion may give rise to the emergence of a foreign T_{H} epitope in the sequence of the analogue, i.e. the emergence of an MHC Class II molecule binding sequence. However, in most situations it is preferable (and even necessary) to introduce a known foreign T_{H} epitope, and such an operation will require amino acid substitution and/or insertion (or sometimes addition in the form of either conjugation to a carrier protein or provision of a fusion polypeptide by means of molecular biology methods). It is preferred that the number of amino acid insertions, deletions, substitutions or additions is at least 2, such as 3, 4, 5, 6, 7, 8, 9, 10, 11, 12, 13, 14, 15, 16, 17, 18, 19, 20, and 25 insertions, substitutions, additions or deletions. It is furthermore preferred that the number of amino acid substitutions is not in excess of 150, such as at most 100, at most 90, at most 80, and at most 70. It is especially preferred that the number of substitutions, insertions, deletions, or additions does not exceed 60, and in particular the number should not exceed 50 or even 40. Most preferred is a number of not more than 36 (corresponding to the number of the total number of amino acids in the P2+P30 epitopes).

The invention requires modification of CEA by introducing at least one foreign immunodominant T_{H} epitope. It will be understood that the question of immune dominance of a T-cell epitope depends on the animal species in question. As used herein, the term “immunodominance” simply refers to epitopes which in the vaccinated individual/population gives rise to a significant immune response, but it is a well-known fact that a T-cell epitope which is immunodominant in one individual is not necessarily immunodominant in another individual of the same species, even though it may be capable of binding MHC-II molecules in the latter individual. True immune dominant T_{H} epitopes are those which, independent of the polypeptide wherein they form a subsequence, give rise to activation of T_{H} cells—in other words, some T_{H} epitopes have, as an intrinsic feature, the characteristic of substantially never being cryptic since they are substantially always processed by APCs and resented in the context of an MHC II molecule on the surface of the APC.

Another important point is the issue of MHC restriction of T-cell epitopes. In general, naturally occurring T-cell epitopes are MHC restricted, i.e. a certain peptides constituting a T-cell epitope will only bind effectively to a subset of MHC Class II molecules. This in turn has the effect that in most cases the use of one specific T-cell epitope will result in a vaccine component which is only effective in a fraction of the population, and depending on the size of that fraction, it can be necessary to include more T_{H} epitopes in the same molecule, or alternatively prepare a multi-component vaccine wherein the components are variants of the antigen, which are distinguished from each other by the nature of the T-cell epitope introduced.

If the MHC restriction of the T-cells used is completely unknown (for instance in a situation where the vaccinated animal has a poorly defined MHC composition), the fraction of the animal population covered by a specific vaccine composition can be determined by means of the following formula:

$${f}_{population} = 1 - {\prod }_{i = 1}^{n} ( 1 - {p}_{i} ) \qquad ( II )$$

where pᵢ is the frequency in the population of responders to the i^{th} foreign T-cell epitope present in the vaccine composition, and n is the total number of foreign T-cell epitopes in the vaccine composition. Thus, a vaccine composition containing 3 foreign T-cell epitopes having response frequencies in the population of 0.8, 0.7, and 0.6, respectively, would give
   1−0.2×0.3×0.4=0.976
i.e. 97.6 percent of the population will statistically mount an MHC-II mediated response to the vaccine.

The above formula does not apply in situations where a more or less precise MHC restriction pattern of the peptides used is known. If, for instance a certain peptide only binds the human MHC-II molecules encoded by HLA-DR alleles DR1, DR3, DR5, and DR7, then the use of this peptide together with another peptide which binds the remaining MHC-II molecules encoded by HLA-DR alleles will accomplish 100% coverage in the population in question. Likewise, if the second peptide only binds DR3 and DR5, the addition of this peptide will not increase the coverage at all. If one bases the calculation of population response purely on MHC restriction of T-cell epitopes in the vaccine, the fraction of the population covered by a specific vaccine composition can be determined by means of the following formula:

$${f}_{population} = 1 - {\prod }_{j = 1}^{3} {( 1 - {φ}_{j} )}^{2} \qquad ( III )$$

wherein φⱼ is the sum of frequencies in the population of allelic haplotypes encoding MHC molecules which bind any one of the T-cell epitopes in the vaccine and which belong to the j^{th} of the 3 known HLA loci (DP, DR and DQ); in practice, it is first determined which MHC molecules will recognize each T-cell epitope in the vaccine and thereafter these MHC molecules are listed by type (DP, DR and DQ)—then, the individual frequencies of the different listed allelic haplotypes are summed for each type, thereby yielding φ₁, φ₂, and φ₃.

It may occur that the value pᵢ in formula II exceeds the corresponding theoretical value πᵢ:

$${π}_{i} = 1 - {\prod }_{j = 1}^{3} {( 1 - {v}_{j} )}^{2} \qquad ( IV )$$

wherein vⱼ is the sum of frequencies in the population of allelic haplotypes encoding MHC molecules which bind the i^{th} T-cell epitope in the vaccine and which belong to the j^{th} of the 3 known HLA loci (DP, DR and DQ). This means that in 1−πᵢ of the population there is a frequency of responders of f_{residual}_{_{—}}ᵢ=(pᵢ−πᵢ)/(1−πᵢ). Therefore, formula III can be adjusted so as to yield formula V:

$${f}_{population} = 1 - {\prod }_{j = 1}^{3} {( 1 - {φ}_{j} )}^{2} + ( 1 - {\prod }_{i = 1}^{n} ( 1 - {f}_{residual\_i} ) ) \qquad ( V )$$
where the term 1−f_{residual}_{_{—}}ᵢ is set to zero if negative. It should be noted that formula V requires that all epitopes have been haplotype mapped against identical sets of haplotypes.

Therefore, when selecting T-cell epitopes to be introduced in the modified CEA polypeptide, it is important to include all knowledge of the epitopes which is available: 1) The frequency of responders in the population to each epitope, 2) MHC restriction data, and 3) frequency in the population of the relevant haplotypes.

There exist a number of naturally occurring “promiscuous” T-cell epitopes which are active in a large proportion of individuals of an animal species or an animal population and these are preferably introduced in the vaccine thereby reducing the need for a very large number of different modified CEAs in the same vaccine.

The promiscuous epitope can according to the invention be a naturally occurring human T-cell epitope such as epitopes from tetanus toxoid (e.g. the P2 and P30 epitopes, cf. SEQ ID NOs: 13 and 14, respectively), diphtheria toxoid, Influenza virus hemagluttinin (HA), and P. falciparum CS antigen.

Over the years a number of other promiscuous T-cell epitopes have been identified. Especially peptides capable of binding a large proportion of HLA-DR molecules encoded by the different HLA-DR alleles have been identified and these are all possible T-cell epitopes to be introduced in modified CEA used according to the present invention. Cf. also the epitopes discussed in the following references which are hereby all incorporated by reference herein: WO 98/23635 (Frazer I H et al., assigned to The University of Queensland); Southwood S et. al, 1998, J. Immunol. 160: 3363-3373; Sinigaglia F et al., 1988, Nature 336: 778-780; Rammensee H G et al., 1995, Immunogenetics 41: 4 178-228; Chicz R M et al., 1993, J. Exp. Med 178: 27-47; Hammer J et al., 1993, Cell 74: 197-203; and Falk K et al., 1994, Immunogenetics 39: 230-242. The latter reference also deals with HLA-DQ and -DP ligands. All epitopes listed in these 5 references are relevant as candidate natural epitopes to be used in the present invention, as are epitopes which share common motifs with these.

Alternatively, the epitope can be any artificial T-cell epitope which is capable of binding a large proportion of haplotypes. In this context the pan DR epitope peptides (“PADRE”) described in WO 95/07707 and in the corresponding paper Alexander J et al., 1994, Immunity 1: 751-761 (both disclosures are incorporated by reference herein) are interesting candidates for epitopes to be used according to the present invention. It should be noted that the most effective PADRE peptides disclosed in these papers carry D-amino acids in the C- and N-termini in order to improve stability when administered. However, the present invention primarily aims at incorporating the relevant epitopes as part of the modified CEA which should then subsequently be broken down enzymatically inside the lysosomal compartment of APCs to allow subsequent presentation in the context of an MHC-II molecule and therefore it is not expedient to incorporate D-amino acids in the epitopes used in the present invention.

One especially preferred PADRE peptide is the one having the amino acid sequence AKFVAAWTLKAAA (SEQ ID NO: 15) or an immunologically effective subsequence thereof. This, and other epitopes having the same lack of MHC restriction are preferred T-cell epitopes which should be present in the modified CEA used in the inventive method. Such super-promiscuous epitopes will allow for the most simple embodiments of the invention wherein only one single modified CEA is presented to the vaccinated animal's immune system.

The nature of the above-discussed variation/modification preferably also comprises that
at least one first moiety is included in the modified CEA, said first moiety effecting targeting of the modified CEA to an antigen presenting cell (APC), and/or
at least one second moiety is included in the modified CEA, said second moiety stimulating the immune system, and/or
at least one third moiety is included in the modified CEA, said third moiety optimising presentation thereof to the immune system.

The functional and structural features relating these first, second and third moieties will be discussed in the following, but also the general description of such moieties given in the “Background of the Invention” section above applies here:

They can be present in the form of side groups attached covalently or non-covalently to suitable chemical groups in the amino acid sequence of the autologous CEA or a subsequence thereof. This is to mean that stretches of amino acid residues derived from the autologous CEA are derivatized without altering the primary amino acid sequence, or at least without introducing changes in the peptide bonds between the individual amino acids in the chain.

The moieties can also be in the form of fusion partners to the amino acid sequence derived from the autologous CEA. In this connection it should be mentioned that both possibilities include the option of conjugating the amino acid sequence to a carrier, cf. the discussion of these below. In other words, in the present context the term “fusion protein is not merely restricted to a fusion construct prepared by means of expression of a DNA fragment encoding the construct but also to a conjugate between two proteins which are joined by means of a peptide bond in a subsequent chemical reaction.

As mentioned above, the modified CEA can also include the introduction of a first moiety which targets the modified CEA to an APC or a B-lymphocyte. For instance, the first moiety can be a specific binding partner for a B-lymphocyte specific surface antigen or for an APC specific surface antigen. Many such specific surface antigens are known in the art. For instance, the moiety can be a carbohydrate for which there is a receptor on the B-lymphocyte or the APC (e.g. mannan or mannose). Alternatively, the second moiety can be a hapten. Also an antibody fragment which specifically recognizes a surface molecule on APCs or lymphocytes can be used as a first moiety (the surface molecule can e.g. be an FCγ receptor of macrophages and monocytes, such as FCγ RI or, alternatively any other specific surface marker such as CD40 or CTLA-4). It should be noted that all these exemplary targeting molecules can be used as part of an adjuvant, cf. below. CD40 ligand, antibodies against CD40, or variants thereof which bind CD40 will target the modified CEA to dendritic cells. At the same time, recent results have shown that the interaction with the CD40 molecule renders the T_{H} cells unessential for obtaining a CTL response. Hence, it is contemplated that the general use of CD40 binding molecules as the first moiety (or as adjuvants, cf. below) will enhance the CTL response considerably; in fact, the use of such CD40 binding molecules as adjuvants and “first moieties” in the meaning of the present invention is believed to be inventive in its own right.

As an alternative or supplement to targeting the modified CEA to a certain cell type in order to achieve an enhanced immune response, it is possible to increase the level of responsiveness of the immune system by including the above-mentioned second moiety, which stimulates the immune system. Typical examples of such second moieties are cytokines, heat-shock proteins, and hormones, as well as effective parts thereof.

Suitable cytokines to be used according to the invention are those which will normally also function as adjuvants in a vaccine composition, e.g. interferon γ (IFN-γ), Flt3 ligand (Flt3L), interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF); alternatively, the functional part of the cytokine molecule may suffice as the second moiety. With respect to the use of such cytokines as adjuvant substances, cf. the discussion below.

Alternatively, the second moiety can be a toxin, such as listeriolycin (LLO), lipid A and heat-labile enterotoxin. Also, a number of mycobacterial derivatives such as MDP (muramyl dipeptide), CFA (complete Freund's adjuvant) and the trehalose diesters TDM and TDE are interesting possibilities.

According to the invention, suitable heat shock proteins used as the second moiety can be HSP70, HSP90, HSC70 (a heat shock cognate), GRP94, and calreticulin (CRT).

Also the possibility of introducing a third moiety that enhances the presentation of the modified CEA to the immune system is an important embodiment of the invention. The art has shown several examples of this principle. For instance, it is known that the palmitoyl lipidation anchor in the Borrelia burgdorferi protein OspA can be utilised so as to provide self-adjuvating polypeptides (cf. e.g. WO 96/40718). It seems that the lipidated proteins form up micelle-like structures with a core consisting of the lipidation anchor parts of the polypeptides and the remaining parts of the molecule protruding therefrom, resulting in multiple presentations of the antigenic determinants. Hence, the use of this and related approaches using different lipidation anchors (e.g. a myristyl group, a farnesyl group, a geranyl-geranyl group, a GPI-anchor, and an N-acyl diglyceride group) are preferred embodiments of the invention, especially since the provision of such a lipidation anchor in a recombinantly produced protein is fairly straightforward and merely requires use of e.g. a naturally occurring signal sequence as a fusion partner for the modified CEA, cf. below. Another possibility is use of the C3d fragment of complement factor C3 or C3 itself (cf. Dempsey et al., 1996, Science 271, 348-350 and Lou & Kohler, 1998, Nature Biotechnology 16, 458-462).

In the present context it is of high importance to note that CEA naturally has a GPI anchor, meaning that by preserving this in the modified molecule, the self-adjuvating effect would be a possibility. The preservation can e.g. be obtained by preserving the natural C-terminal signal sequence from the native CEA encoding sequence (the C-terminal signal sequence spans amino acids 648 through 668 in SEQ ID NO: 2, meaning that the mature CEA spans amino acids 1-647 in SEQ ID NOs: 2 and 4). By including this known signal sequence also in constructs that do not include the C-terminus of CEA, it will be achieved that the resulting expression product is anchored to the membrane.

However, it is known that expression of membrane bound proteins may be at a lower level than that of a secreted protein, so also modified versions of CEA that lack the GPI anchor are embraced by the present invention.

It is important to note that when attempting to use the method of the invention against epitopes of the extracellularly exposed parts of CEA, it is most preferred that the modified CEA substantially preserves the 3-dimensional structure of one or more domains of CEA. Thus, in the present specification and claims this is intended to mean that the 3-dimensional structure of the part of CEA which is extracellularly exposed is preserved, since, as mentioned above, part of CEA are not expected to effectively engage the humeral immune system. It is in this context preferred that the 3D structures of at least 4 domains are substantially preserved, especially domains 1-4.

In a particularly preferred embodiment, the 3D structures of all 7 domains are substantially preserved.

For the purposes of the present invention, it is however sufficient if the variation/modification (be it an insertion, addition, deletion or substitution) gives rise to a foreign T-cell epitope and at the same time preserves a substantial number of the CTL epitopes in CEA (and sometimes also a substantial number of B-cell epitopes).

The following formula describes the constructs generally covered by the invention:
(MOD₁)ₛ₁(CEAₑ₁)ₙ₁(MOD₂)^{s2}(CEAₑ₂)ₙ₂ . . . (MODₓ)ₛₓ(CEAₑₓ)ₙₓ (I)
where CEAₑ₁−CEAₑₓ are x CTL and/or B-Cell epitope containing subsequences of the autologous CEA which independently are identical or non-identical and which may contain or not contain foreign side groups, x is an integer ≧3, n1-nx are x integers ≧0 (at least one is ≧1), MOD₁-MODₓ are x modifications introduced between the preserved epitopes, and s1-sx are x integers ≧0 (at least one is >1 if no side groups are introduced in the sequences). Thus, given the general functional restraints on the immunogenicity of the constructs as well as the requirements set forth above, the invention allows for all kinds of permutations of the original constant CEA sequence, and all kinds of modifications therein. Thus, included in the invention are modified CEA obtained by omission of parts of the autologous CEA sequence.

It is furthermore preferred that the variation and/or modification includes duplication, when applicable, of the at least one B-cell epitope, or of at least one CTL epitope of the autologous CEA. This strategy will give the result that multiple copies of preferred epitopic regions are presented to the immune system and thus maximizing the probability of an effective immune response. Hence, this embodiment of the invention utilises multiple presentations of epitopes derived from the autologous CEA (i.e. formula I wherein at least one B-cell epitope is present in two positions).

This effect can be achieved in various ways, e.g. by simply preparing fusion polypeptides comprising the structure (CEAₑ)ₘ, where m is an integer ≧2 and CEAₑ is a region of constant CEA heavy or light chain containing at least one CTL or B-cell epitope and then introduce the modifications discussed herein in at least one of the epitope containing sequences.

An alternative embodiment of the invention which also results in the preferred presentation of multiple (e.g. at least 2) copies of the important epitopic regions of the autologous CEA to the immune system is the covalent coupling of the autologous CEA, subsequence or variants thereof to certain molecules. For instance, polymers can be used, e.g. carbohydrates such as dextran, cf. e.g. Lees A et al., 1994, Vaccine 12: 1160-1166; Lees A et al., 1990, J. Immunol. 145: 3594-3600, but also mannose and mannan are useful alternatives. Integral membrane proteins from e.g. E. coli and other bacteria are also useful conjugation partners. The traditional carrier molecules such as keyhole limpet hemocyanin (KLH), tetanus toxoid, diphtheria toxoid, and bovine serum albumin (BSA) are also preferred and useful conjugation partners.

Preservation of the sometimes advantageous substantial fraction of B-cell epitopes or even the 3D structure of autologous CEA (or domains thereof) which is subjected to modification as described herein can be confirmed in several ways. One is simply to prepare a polyclonal antiserum directed against the autologous CEA (e.g. an antiserum prepared in a rabbit or another suitable animal) and thereafter use this antiserum as a test reagent (e.g. in a competitive ELISA) against the modified proteins which are produced. Modified versions (analogues) which react to the same extent with the antiserum as does the autologous CEA must be regarded as having the same 3D structure as the autologous CEA whereas modified CEA exhibiting a limited (but still significant and specific) reactivity with such an antiserum is regarded as having maintained a substantial fraction of the original B-cell epitopes.

Alternatively, a selection of monoclonal antibodies reactive with distinct epitopes on the autologous CEA can be prepared and used as a test panel. This approach has the advantage of allowing 1) an epitope mapping of the autologous CEA and 2) a mapping of the epitopes which are maintained in the modified CEA prepared.

Of course, a third approach would be resolve the 3-dimensional structure of the autologous CEA or of a biologically active truncate thereof (cf. above) and compare this to the resolved three-dimensional structure of the modified CEA prepared. Three-dimensional structure can be resolved by the aid of X-ray diffraction studies and NMR-spectroscopy. Further information relating to the tertiary structure can to some extent be obtained from circular dichroism studies which have the advantage of merely requiring the polypeptide in pure form (whereas X-ray diffraction requires the provision of crystallized polypeptide and NMR requires the provision of isotopic variants of the polypeptide) in order to provide useful information about the tertiary structure of a given molecule. However, ultimately X-ray diffraction and/or NMR are necessary to obtain conclusive data since circular dichroism can only provide indirect evidence of correct 3-dimensional structure via information of secondary structure elements.

The present invention relies on the identification of especially well-suited regions for introduction of the foreign element that must provide for the necessary T_{H} epitopes. Especially preferred regions are flexible loop regions (which do not contribute directly to tertiary structure) as well as flexible linker regions and N or C termini. Alternatively, the introduction of the T_{H} epitope can be made in a region that has a secondary structure that has a high degree of similarity with the secondary structure of the epitope (an α-helical region may be substituted with an α-helical epitope, a β-sheet region may be substituted with a β-sheet containing epitope etc).

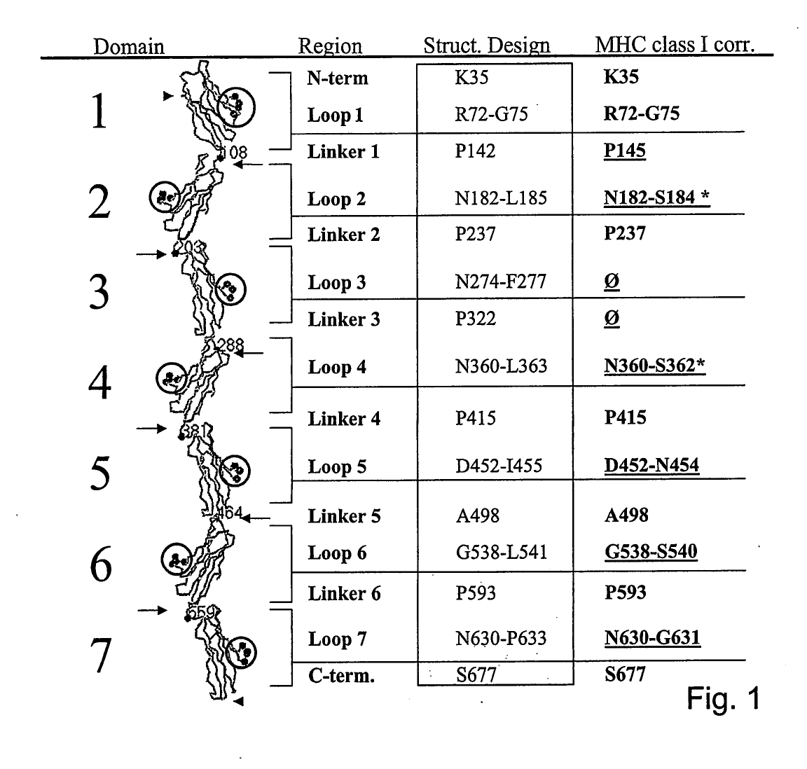

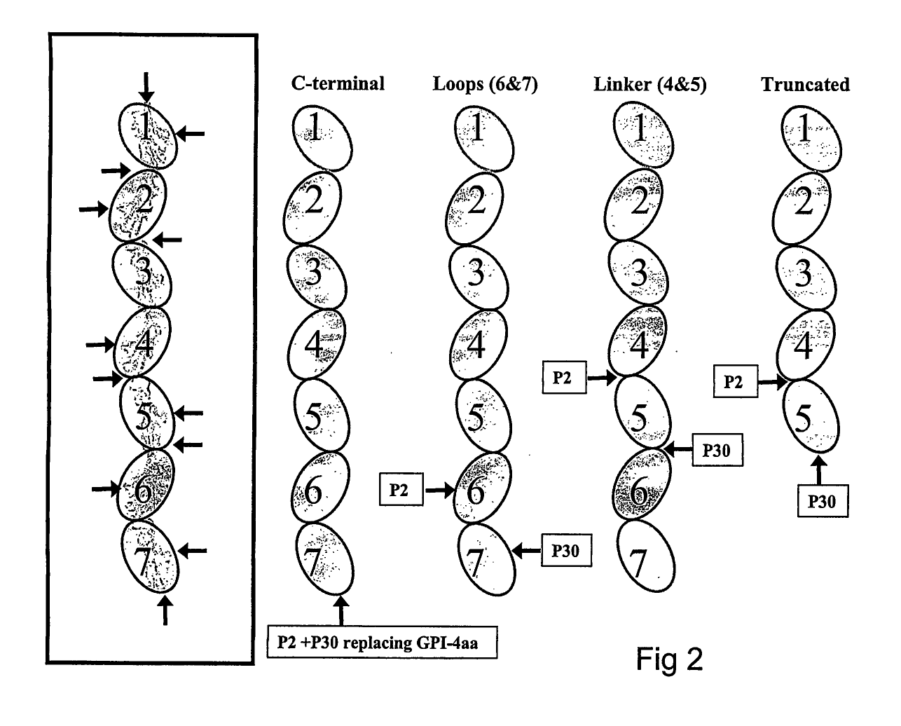

Especially preferred modified CEA polypeptides useful in the present invention are selected from the group consisting of those where the T_{H} epitope is introduced: in the C-terminus (some variants will delete the GPI anchor as a consequence of the omission of the native signal sequence); in the loop structures in any one of domains 1-7 as shown in FIG. 1; and between any two adjacent domains in CEA.

It is important to note that when a CEA construct is prepared by amino acid substitution with a foreign epitope, the introduction is supposed to influence minimally on the epitopes in the relevant CEA fragment. Hence, normally a substitution will only result in a CEA variant where the deleted CEA amino acids constitute 30% or less of the relevant CEA (sub)sequence, and under normal circumstances this number will be much lower such as at most 20%, at most 15%, at most 10%, and at most 7.5%. Being a large molecule, the number can be even lower, such as at most 5%, at most 4% and even as little as at most 3% or at most 2%.

Especially preferred modified CEA polypeptides will contain introduction of the foreign T_{H} epitope as an addition to the C- or N-terminus of mature CEA (i.e. before amino acid 35 or after amino acid 681); as an insertion before any one of CEA amino acids 1, 38, 39, 40, 41, 42, 111, 148, 149, 150, 151, 203, 326, 327, 328, 329, 381, 418, 419, 420, 421, 464, 504, 505, 506, 507, 559, 596, 597, 598, and 643; as a substitution that includes deletion of any one or all of amino acids 38, 39, 40, and 41; as a substitution that includes deletion of any or all of amino acids 148, 149, and 150; as a substitution that includes deletion of any one or all of amino acids 326, 327, and 328; as a substitution that includes deletion of any or all of amino acids 418, 419, and 420; as a substitution that includes deletion of any or all of amino acids 504, 505, and 506; and as a substitution that includes deletion of any one or all of amino acids 596 and 597. All amino acid numbering corresponds to that of SEQ ID NO: 2.

Particularly preferred modified CEA polypeptides are set forth in the sequence listing in SEQ ID NOs: 6, 8, 10, and 12, and are also shown schematically in FIG. 2—all these constructs are made without the native CEA signal sequence and are therefore not membrane anchored—according to the invention it is nevertheless intended that these 4 preferred variants (and all other CEA variants disclosed herein) may be produced from genetic material that includes this signal sequence and thereby the variants become membrane anchored.

SEQ ID NO: 6 sets forth the amino acid sequence of a polypeptide that introduces the tetanus toxoid P2 and P30 epitopes in the C-terminus of CEA. The amino acid sequence in SEQ ID NO: 8 sets forth a polypeptide that introduces the P2 and P30 epitopes in the loops of domains 6 and 7, respectively. SEQ ID NO: 10 sets forth the amino acid sequence of a polypeptide that includes the P2 and P30 epitopes inserted in the linker regions between domains 4 and 5 as well as 5 and 6, respectively. Finally, SEQ ID NO: 12 sets forth the amino acid sequence of a modified CEA polypeptide where the P2 epitope is inserted in the linker between domains 4 and 5 and where P30 is substitutes domains 6 and 7.

In essence, there are at present three feasible ways of obtaining the presentation of the relevant epitopes to the immune system: Traditional sub-unit vaccination with polypeptide antigens, administration of a genetically modified live vaccine/viral vector, and nucleic acid vaccination. These three possibilities will be discussed separately in the following:

Polypeptide Vaccination

This entails administration to the animal in question of an immunogenically effective amount of the at least modified CEA polypeptide, e.g. as the above-discusse 1^{st} and/or 2^{nd} analogues. Preferably, the at least one modified CEA polypeptide is formulated together with a pharmaceutically and immunologically acceptable carrier and/or vehicle and, optionally an adjuvant.

When effecting presentation of the modified CEA to an animal's immune system by means of administration thereof to the animal, the formulation of the polypeptide follows the principles generally acknowledged in the art.

Preparation of vaccines which contain peptide sequences as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such vaccines are prepared as injectables either as liquid solutions or suspensions; solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines; cf. the detailed discussion of adjuvants below.

The vaccines are conventionally administered parenterally, by injection, for example, either subcutaneously, intracutaneously, or intramuscularly. Additional formulations which are suitable for other modes of administration include suppositories and, in some cases, oral, buccal, sublingual, intraperitoneal, intravaginal, anal and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10-95% of active ingredient, preferably 25-70%. For oral formulations, cholera toxin is an interesting formulation partner (and also a possible conjugation partner).

The modified CEA may be formulated into the vaccine as neutral or salt forms. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2000 μg (even though higher amounts in the 1-10 mg range are contemplated), such as in the range from about 0.5 μg to 1000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations. The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the polypeptides of the vaccine are sufficiently immunogenic in a vaccine, but for some of the others the immune response will be enhanced if the vaccine further comprises an adjuvant substance. It is especially preferred to use an adjuvant which can be demonstrated to facilitate breaking of the autotolerance to autoantigens.

Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

One group of preferred adjuvants facilitate uptake of the vaccine molecules by APCs, such as dendritic cells, and activate these. Non-limiting examples are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant. In general it should be noted that the disclosures above which relate to compounds and agents useful as first, second and third moieties in the modified CEA also refer mutatis mutandis to their use in the adjuvant of a vaccine of the invention.

The application of adjuvants include use of agents such as aluminium hydroxide or phosphate (alum), commonly used as 0.05 to 0.1 percent solution in buffered saline, admixture with synthetic polymers of sugars (e.g. Carbopol®) used as 0.25 percent solution, aggregation of the protein in the vaccine by heat treatment with temperatures ranging between 700 to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

According to the invention DDA (dimethyldioctadecylammonium bromide) is an interesting candidate for an adjuvant as is DNA, MF59, and γ-inulin, but also Freund's complete and incomplete adjuvants as well as quillaja saponins such as QuilA and QS21 are interesting. Further possibilities are monophosphoryl lipid A (MPL), and the above mentioned C3 and C3d.

Liposome formulations are also known to confer adjuvant effects, and therefore liposome adjuvants are preferred according to the invention.

Also immunostimulating complex matrix type (ISCOM® matrix) adjuvants are preferred choices according to the invention, especially since it has been shown that this type of adjuvants are capable of up-regulating MHC Class II expression by APCS. An ISCOM® matrix consists of (optionally fractionated) saponins (triterpenoids) from Quillaja saponaria, cholesterol, and phospholipid. When admixed with the immunogenic protein, the resulting particulate formulation is what is known as an ISCOM particle where the saponin constitutes 60-70% w/w, the cholesterol and phospholipid 10-15% w/w, and the protein 10-15% w/w. Details relating to composition and use of immunostimulating complexes can e.g. be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

Another highly interesting (and thus, preferred) possibility of achieving adjuvant effect is to employ the technique described in Gosselin et al., 1992 (which is hereby incorporated by reference herein). In brief, the presentation of a relevant antigen such as a modified CEA polypeptide of the present invention can be enhanced by conjugating the antigen to antibodies (or antigen binding antibody fragments) against the Fcγ receptors on monocytes/macrophages. Especially conjugates between modified CEA and anti-FcγRI have been demonstrated to enhance immunogenicity for the purposes of vaccination.

Other possibilities involve the use of the targeting and immune modulating substances (i.a. cytokines) mentioned above as candidates for the first and second moieties in the modified CEA. In this connection, also synthetic inducers of cytokines like poly I:C are possibilities.

Suitable mycobacterial derivatives are selected from the group consisting of muramyl dipeptide, complete Freund's adjuvant, RIBI, and a diester of trehalose such as TDM and TDE.

Suitable immune targeting adjuvants are selected from the group consisting of CD40 ligand and CD40 antibodies or specifically binding fragments thereof (cf. the discussion above), mannose, a Fab fragment, and CTLA-4.

Suitable polymer adjuvants are selected from the group consisting of a carbohydrate such as dextran, PEG, starch, mannan, and mannose; a plastic polymer; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is i.a. described briefly in Gelber C et al., 1998, “Elicitation of Robust Cellular and Humeral Immune Responses to Small Amounts of Immunogens Using a Novel Medical Device Designated the Virtual Lymph ode”, in: “From the Laboratory to the Clinic, Book of Abstracts, October 12^{th}-15^{th} 1998, Seascape Resort, Aptos, California”.

At any rate, for all (poly)peptide vaccine formulations according to the invention, it is important that, if a CTL response is aimed at, the formulation is capable of shunting the polypeptide immunogen into the MHC type I degradation pathway in order to ensure that the CTL epitopes of autologous CEA are presented in the context of MHC Class I molecules on the surface of the APC. The skilled person will know which of the above-detailed adjuvants to choose for this specific purpose.

It is expected that the vaccine should be administered at least once a year, such as at least 1, 2, 3, 4, 5, 6, and 12 times a year. More specifically, 1-12 times per year is expected, such as 1, 2, 3, 4, 5, 6, 7, 8, 9, 10, 11, or 12 times a year to an individual in need thereof. It has previously been shown that the memory immunity induced by the use of the preferred autovaccines according to the invention is not permanent, and therefore the immune system needs to be periodically challenged with the modified CEA.

Due to genetic variation, different individuals may react with immune responses of varying strength to the same polypeptide. Therefore, the vaccine according to the invention may comprise several different modified CEA polypeptides in order to increase the immune response, cf. also the discussion above concerning the choice of foreign T-cell epitope introductions. The vaccine may comprise two or more polypeptides, where all of the polypeptides are as defined above.

The vaccine may consequently comprise 3-20 different modified polypeptides, such as 3-10 different polypeptides. However, normally the number of peptides will be sought kept to a minimum such as 1 or 2 peptides.

Live Vaccines

The second alternative for effecting presentation to the immune system is the use of live vaccine technology. In live vaccination, presentation to the immune system is effected by administering, to the animal, a non-pathogenic microorganism which has been transformed with a nucleic acid fragment encoding the necessary epitopic regions or a complete modified CEA polypeptide (e.g. a 1^{st} and/or 2^{nd} analogue). Alternatively, the microorganism is transformed with a vector incorporating such a nucleic acid fragment. The non-pathogenic microorganism can be any suitable attenuated bacterial strain (attenuated by means of passaging or by means of removal of pathogenic expression products by recombinant DNA technology), e.g. Mycobacterium bovis BCG., non-pathogenic Streptococcus spp., E. coli, Salmonella spp., Vibrio cholerae, Shigella, etc. Reviews dealing with preparation of state-of-the-art live vaccines can e.g. be found in Saliou P, 1995, Rev. Prat. 45: 1492-1496 and Walker P D, 1992, Vaccine 10: 977-990, both incorporated by reference herein. For details about the nucleic acid fragments and vectors used in such live vaccines, cf. the discussion below.

Especially BCG has been used extensively as a live bacterial vaccine: The BCG vaccine has been successfully used to prevent tuberculosis around the world. Vaccination can be given after birth and results in few severe complications, even in individuals who are infected with human immunodeficiency virus type 1. BCG possesses strong immune adjuvant activity, and has been used extensively in the treatment of superficial bladder cancers.

As for the polypeptide vaccine, the T_{H} epitope and/or the first and/or second and/or third moieties can, if present, be in the form of fusion partners to the amino acid sequence derived from the autologous CEA.

As an alternative to bacterial live vaccines, the nucleic acid fragment of the invention discussed below can be incorporated in a non-virulent viral vaccine vector. Feasible viral vectors are selected from a pox virus such as vaccinia, MVA (modified Vaccinia virus), canary pox, avi-pox, and chicken pox etc. Alternatively, a herpes simplex virus variant can be used.

When using viral vaccines it is an interesting embodiment of the invention to utilise expression vectors where the N-terminal signal sequence (amino acids 1-34 in SEQ ID NO: 2) is deleted by omitting the nucleotides encoding it. This will have as a result that the translation product is not exported to the ER in the host cell, hence facilitating presentation of CTL epitopes on the surface of infected cells, cf. also the discussion under “nucleic acid vaccination”.

Normally, the non-pathogenic microorganism or virus is administered only once to the animal, but in certain cases it may be necessary to administer the microorganism more than once in a lifetime.

Also, the microorganism can be transformed with nucleic acid(s) containing regions encoding the 1^{st}, 2^{nd} and/or 3^{rd} moieties, e.g. in the form of the immunomodulating substances described above such as the cytokines discussed as useful adjuvants. A preferred version of this embodiment encompasses having the coding region for the modified CEA and the coding region for the immunomodulator in different open reading frames or at least under the control of different promoters. Thereby it is avoided that the modified CEA polypeptides or epitopes are produced as fusion partners to the immunomodulator. Alternatively, two distinct nucleotide fragments can be used as transforming agents.

Nucleic Acid Vaccination

As an alternative to classic administration of a peptide-based vaccine, the technology of nucleic acid vaccination (also known as “nucleic acid immunisation”, “genetic immunisation”, “gene immunisation” and “DNA vaccination) offers a number of attractive features.

First, in contrast to the traditional vaccine approach, nucleic acid vaccination does not require resource consuming large-scale production of the immunogenic agent (e.g. in the form of industrial scale fermentation of microorganisms producing the modified CEA polypeptides necessary in polypeptide vaccination). Furthermore, there is no need to device purification and refolding schemes for the immunogen. And finally, since nucleic acid vaccination relies on the biochemical apparatus of the vaccinated individual in order to produce the expression product of the nucleic acid introduced, the optimum posttranslational processing of the expression product is expected to occur; this is especially important in the case of autovaccination, since, as mentioned above, a significant fraction of the original B-cell epitopes should be preserved in the modified CEA polypeptides derived from extracellularly exposed polypeptide sequences, and since B-cell epitopes in principle can be constituted by parts of any (bio)molecule (e.g. carbohydrate, lipid, protein etc.). Therefore, native glycosylation and lipidation patterns of the immunogen may very well be of importance for the overall immunogenicity and this is best ensured by having the host producing the immunogen.

Two further features render nucleic acid vaccination especially interesting in the context of the present invention. By using DNA as a vaccine agent, it is relatively uncomplicated to ensure presentation of CTL epitopes in the MHC class I context on the APCs (as long as translocation signals directing the translation product to the ER are omitted). Further, it has been repeatedly demonstrated that immunizations including administration of DNA leads to a shift in T helper cell profile from Th2 to Th1 cells, and since the adverse allergic reactions mediated by CEA are first and foremost supported by Th2 cells, the use of DNA vaccination will in itself provide a beneficial effect on the underlying disease.

Hence, an important embodiment of the method of the invention involves that presentation is effected by administering a nucleic acid fragment encoding a modified CEA polypeptide. Preferably, this is done by using a nucleic acid fragment which encodes and expresses the above-discussed first analogue. If the first analogue is equipped with the above-detailed T_{H} epitopes and/or first and/or second and/or third moieties, these are then present in the form of fusion partners to the amino acid sequence derived from the autologous CEA, the fusion construct being encoded by the nucleic acid fragment.

As for the traditional vaccination approach, the nucleic acid vaccination can be combined with in vivo introduction, into the APC, of at least one nucleic acid fragment encoding and expressing the second analogue. The considerations pertaining to 1^{st}, 2^{nd} and 3^{rd} moieties and T_{H} epitopes apply also here.

In this embodiment, the introduced nucleic acid is preferably DNA which can be in the form of naked DNA, DNA formulated with charged or uncharged lipids, DNA formulated in liposomes, emulsified DNA, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, and DNA formulated with an adjuvant. In this context it is noted that practically all considerations pertaining to the use of adjuvants in traditional vaccine formulation apply for the formulation of DNA vaccines. Hence, all disclosures herein which relate to use of adjuvants in the context of polypeptide based vaccines apply mutatis mutandis to their use in nucleic acid vaccination technology. The same holds true for other considerations relating to formulation and mode and route of administration and, hence, also these considerations discussed above in connection with a traditional vaccine apply mutatis mutandis to their use in nucleic acid vaccination technology.

One especially preferred type of formulation of nucleic acid vaccines are microparticles containing the DNA. Suitable microparticles are e.g. described in WO 98/31398.

Furthermore, the nucleic acid(s) used as an immunization agent can contain regions encoding the 1^{st}, 2^{nd} and/or 3^{rd} moieties, e.g. in the form of the immunomodulating substances described above such as the cytokines discussed as useful adjuvants. A preferred version of this embodiment encompasses having the coding region for the modified CEA and the coding region for the immunomodulator in different open reading frames or at least under the control of different promoters. Thereby it is avoided that the modified CEA is produced as a fusion partner to the immunomodulator. Alternatively, two distinct nucleotide fragments can be used, but this is less preferred because of the advantage of ensured co-expression when having both coding regions included in the same molecule.

Under normal circumstances, the nucleic acid of the vaccine is introduced in the form of a vector wherein expression is under control of a viral promoter. For more detailed discussions of vectors according to the invention, cf. the discussion below. Also, detailed disclosures relating to the formulation and use of nucleic acid vaccines are available, cf. Donnelly J J et al, 1997, Annu. Rev. Immunol. 15: 617-648 and Donnelly J J et al., 1997, Life Sciences 60: 163-172. Both of these references are incorporated by reference herein.

The expression cassette in the nucleic acid vaccine can be constructed so as to ensure that no export of the expression product takes place (e.g. by omitting signal sequences that would result in membrane integration or secretion). In this way, only minute amounts of expression product will be exported, whereas the remainder will be processed and presented as peptide fragments in the context of MHC molecules.

Hence, when using nucleic acid vaccines it is an interesting embodiment of the invention to utilise expression vectors where the N-terminal CEA signal sequence (amino acids 1-34 in SEQ ID NO: 2) is deleted by omitting the nucleotides encoding it. This will have as a result that the translation product is not exported to the ER in the host cell, hence facilitating presentation of CTL epitopes on the surface of infected cells. In this way, only minute amounts of expression product will be exported, whereas the remainder will be processed and presented as peptide fragments in the context of MHC molecules. Hence, no or only a very limited antibody response will be induced, whereas a CTL response will be mounted.

Combination of Approaches

As discussed above under the “Background of the Invention”, various prime-boost strategies have proven effective in facilitating an improved immune response. Hence, according to the present invention, use of any combination of nucleic acid vaccination, live vaccination and polypeptide vaccination may be utilised. However, it is especially preferred to prime via nucleic acid or viral vaccination and boost with a polypeptide vaccine, preferably where the polypeptide vaccine contains the expression product front the nucleic acid of the priming vaccine.

Polypeptides of the Invention

All modified CEA polypeptides discussed in the above disclosure of the method of the invention are believed to be inventive in their own right. Hence the invention also relates to modified human CEA polypeptide that is capable of inducing an immune response against autologous CEA in a human subject, the, which comprises at least about 80 CEA-derived amino acids, either in the form of at least about 80 consecutive CEA-derived amino acids or in the form of at least about 80 amino acids constituted of uninterrupted CEA-derived CTL epitopes, and at least one first non-human T helper epitope (T_{H} epitope).

All teaching set forth above concerning possible embodiments of the modified CEA also applies mutatis mutandis to the part of the invention that relates to modified CEA as such. Hence, each and every embodiment of modified CEA described in context of the method of the invention also applies to embodiments of modified CEA as such.

It should be noted that preferred modified CEA polypeptides of the invention (and also the relevant modified CEA polypeptides used in the methods of the invention) comprise modifications which results in a polypeptide having a sequence identity of at least 70% with the autologous CEA or with a subsequence thereof of at least 10 amino acids in length. Higher sequence identities are preferred, e.g. at least 75% or even at least 80% or 85%. The sequence identity for proteins and nucleic acids can be calculated as (N_{ref}−N_{dif})×100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref=}8).

Nucleic Acid Fragments and Vectors of the Invention

It will be appreciated from the above disclosure that the modified polypeptides can be prepared by means of recombinant gene technology but also by means of chemical synthesis or semisynthesis; the latter two options are especially relevant when the modification consists in coupling to protein carriers (such as KLH, diphtheria toxoid, tetanus toxoid, and BSA) and non-proteinaceous molecules such as carbohydrate polymers and of course also when the modification comprises addition of side chains or side groups to an polypeptide-derived peptide chain.

For the purpose of recombinant gene technology, and of course also for the purpose of nucleic acid immunization, nucleic acid fragments encoding the necessary modified CEA polypeptides are important chemical products. Hence, an important part of the invention pertains to a nucleic acid fragment which encodes a modified CEA polypeptide described above, preferably a polypeptide wherein has been introduced a foreign T_{H}-cell epitope by means of insertion and/or addition, preferably by means of substitution and/or deletion. The nucleic acid fragments of the invention are either DNA or RNA fragments.

The nucleic acid fragments of the invention will normally be inserted in suitable vectors to form cloning or expression vectors carrying the nucleic acid fragments of the invention; such novel vectors are also part of the invention. Details concerning the construction of these vectors of the invention will be discussed in context of transformed cells and microorganisms below. The vectors can, depending on purpose and type of application, be in the form of plasmids, phages, cosmids, mini-chromosomes, or virus, but also naked DNA which is only expressed transiently in certain cells is an important vector. Preferred cloning and expression vectors of the invention are capable of autonomous replication, thereby enabling high copynumbers for the purposes of high-level expression or high-level replication for subsequent cloning.

The general outline of a vector of the invention comprises the following features in the 5′→3′ direction and in operable linkage: a promoter for driving expression of the nucleic acid fragment of the invention, optionally a nucleic acid sequence encoding a leader peptide enabling secretion of or integration into the membrane of the polypeptide fragment, the nucleic acid fragment of the invention, and a nucleic acid sequence encoding a terminator. When operating with expression vectors in producer strains or cell-lines it is for the purposes of genetic stability of the transformed cell preferred that the vector when introduced into a host cell is integrated in the host cell genome. In contrast, when working with vectors to be used for effecting in vivo expression in an animal (i.e. when using the vector in DNA vaccination) it is for security reasons preferred that the vector is not capable of being integrated in the host cell genome; typically, naked DNA or nonintegrating viral vectors are used, the choices of which are well-known to the person skilled in the art.

The vectors of the invention are used to transform host cells to produce the modified CEA of the invention. Such transformed cells, which are also part of the invention, can be cultured cells or cell lines used for propagation of the nucleic acid fragments and vectors of the invention, or used for recombinant production of the modified CEA of the invention. Alternatively, the transformed cells can be suitable live vaccine strains wherein the nucleic acid fragment (one single or multiple copies) have been inserted so as to effect secretion or integration into the bacterial membrane or cell-wall of the modified CEA.

Preferred transformed cells of the invention are microorganisms such as bacteria (such as the species Escherichia [e.g. E. coli], Bacillus [e.g. Bacillus subtilis], Salmonella, or Mycobacterium [preferably non-pathogenic, e.g. M. bovis BCG]), yeasts (such as Saccharomyces cerevisiae), and protozoans. Alternatively, the transformed cells are derived from a multi-cellular organism such as a fungus, an insect cell, a plant cell, or a mammalian cell. Most preferred are cells derived from a human being, cf. the discussion of cell lines and vectors below.

For the purposes of cloning and/or optimised expression it is preferred that the transformed cell is capable of replicating the nucleic acid fragment of the invention. Cells expressing the nucleic fragment are preferred useful embodiments of the invention; they can be used for small-scale or large-scale preparation of the modified CEA or, in the case of non-pathogenic bacteria, as vaccine constituents in a live vaccine.

When producing the modified CEA of the invention by means of transformed cells, it is convenient, although far from essential, that the expression product is either exported out into the culture medium or carried on the surface of the transformed cell.

When an effective producer cell has been identified it is preferred, on the basis thereof, to establish a stable cell line which carries the vector of the invention and which expresses the nucleic acid fragment encoding the modified CEA. Preferably, this stable cell line secretes or carries the modified CEA of the invention, thereby facilitating purification thereof.

In general, plasmid vectors containing replicon and control sequences which are derived from species compatible with the host cell are used in connection with the hosts. The vector ordinarily carries a replication site, as well as marking sequences which are capable of providing phenotypic selection in transformed cells. For example, E. coli is typically transformed using pBR322, a plasmid derived from an E. coli species (see, e.g., Bolivar et al., 1977). The pBR322 plasmid contains genes for ampicillin and tetracycline resistance and thus provides easy means for identifying transformed cells. The pBR plasmid, or other microbial plasmid or phage must also contain, or be modified to contain, promoters which can be used by the prokaryotic microorganism for expression.

Those promoters most commonly used in recombinant DNA construction include the B-lactamase (penicillinase) and lactose promoter systems (Chang et al., 1978; Itakura et al., 1977; Goeddel et al., 1979) and a tryptophan (trp) promoter system (Goeddel et al., 1979; EP-A-0 036 776). While these are the most commonly used, other microbial promoters have been discovered and utilized, and details concerning their nucleotide sequences have been published, enabling a skilled worker to ligate them functionally with plasmid vectors (Siebwenlist et al., 1980). Certain genes from prokaryotes may be expressed efficiently in E. coli from their own promoter sequences, precluding the need for addition of another promoter by artificial means.

In addition to prokaryotes, eukaryotic microbes, such as yeast cultures may also be used, and here the promoter should be capable of driving expression. Saccharomyces cerevisiase, or common baker's yeast is the most commonly used among eukaryotic microorganisms, although a number of other strains are commonly available such as Pichia pastoris. For expression in Saccharomyces, the plasmid YRp7, for example, is commonly used (Stinchcomb et al., 1979; Kingsman et al., 1979; Tschemper et al., 1980). This plasmid already contains the trpl gene which provides a selection marker for a mutant strain of yeast lacking the ability to grow in tryptophan for example ATCC No. 44076 or PEP4-1 (Jones, 1977). The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate in culture (tissue culture) has become a routine procedure in recent years (Tissue Culture, 1973). Examples of such useful host cell lines are VERO and HeLa cells, Chinese hamster ovary (CHO) cell lines, and W138, BHK, COS-7 293 and MDCK cell lines.

Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication (Fiers et al., 1978). Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replication may be provided either by construction of the vector to include an exogenous origin, such as may be derived from SV40 or other viral (e.g., Polyoma, Adeno, VSV, BPV) or may be provided by the host cell chromosomal replication mechanism. If the vector is integrated into the host cell chromosome, the latter is often sufficient.

Compositions of the Invention

The invention also relates to an immunogenic composition which comprises, as an effective immunogenic agent at least one of the modified CEA polypeptides described herein in admixture with a pharmaceutically and immunologically acceptable carrier, vehicle, diluent, or excipient, and optionally an adjuvant, cf. also the discussion of these entities in the description of the method of the invention above.

Furthermore, the invention also relates to a composition for inducing production of antibodies autologous CEA, the composition comprising
a nucleic acid fragment or a vector of the invention, and
a pharmaceutically and immunologically acceptable diluent and/or vehicle and/or carrier and/or excipient and/or adjuvant.

Formulation and other specifics concerning such compositions are discussed in the relevant section regarding nucleic acid immunisation above.

In the following examples we present a discussion of the preferred constructs of the invention as well as of their preparation and the testing of the immunological properties of these constructs.

### EXAMPLES

CEA DNA Constructions.

Modified CEA constructs are designed and constructed as generally and specifically described above utilising standard protocols known in the art.

For use as DNA vaccines, the DNA encoding modified CEA can thereafter be directly cloned into suitable, commercially available DNA vaccination vectors such as pcDNA, pHP, pCI etc.

Expression and Purification of CEA Proteins

A variety of expression systems could be employed in order to generate recombinant modified CEA polyptides. This includes expression systems based on e.g. bacterial, insect cells, yeast and mammalian cells. In either system, stable lines and/or clones will be established and grown in suitable volumes for protein production. Various protein purification methods (e.g. precipitations and chromatographic methods such as gel filtrations, affinity chromatography, ion exchange chromatography, HPLC etc.) can be used to purify CEA proteins. If necessary, refolding procedures may be also applied to yield products suitable for vaccinations.

DNA and Protein Vaccinations

The modified CEA polypeptide or nucleic acid expression vector is injected into a suitable animal species such as mouse, rat, guinea pig, rabbit, or monkey.

In case of CEA nucleic acid vaccines, the animals are usually immunized a total of 3-6 times, for example at weeks 0, 2, 4, 6 and 8. The DNA vaccines can either consist of plasmid DNA dissolved in water, saline or a suitable buffer such as trisbased buffers, PBS etc—or the DNA can be formulated in a suitable delivery system such as microparticles and liposomes, cf. the discussion pertaining to formulations above.

In case of protein-based CEA vaccines, the purified recombinant modified CEA proteins can be mixed with a suitable adjuvant such as for example Freund's Adjuvant, ISA-51, aluminum-based adjuvants (aluminium phosphate or aluminium hydroxide, e.g. from Danfoss), Calcium Phosphate, QS21 (Antigenics), MF59 (Chiron Corp.), and Ribi (Glaxo SmithKline). Protein vaccines are usually administered 3-5 times, for example at weeks 0, 3, 6, 9, 12.

CEA nucleic acid vaccines and CEA protein vaccines can also be combined in any vaccination scheme in a prime-boost strategy. An example of such a vaccination scheme could be one initial immunisation with a CEA DNA expression vector at week 0 and subsequent booster injections with modified CEA protein at weeks 3, 6 and 9—however, any other combination of DNA and protein vaccinations could be used.

Antibody Titer Determination

Sera from vaccinated animals can be tested for CEA specific antibodies by ELISA. 96-well Maxisorb plates (e.g obtained from Nunc, Life Technologies, Taastrup, Denmark) can be coated with a suitable volume (e.g. 50 ul) of CEA protein in a suitable buffer such as carbonate buffer pH 9.6 in a suitable concentration giving a final CEA content of e.g. 1 ug/well. The plates are incubated, e.g. for 1 hour, washed in washing buffer, e.g. PBS+0.5M NaCl+1% Triton X-100 and then blocked for e.g. 1 hour in dilution buffer that could e.g. be washing buffer plus 1% BSA. Standards and diluted serum samples can be added in duplicate and incubated in the plates, e.g. for 30 minutes. After washing, a dilution of secondary antibody (e.g. HRP conjugated rabbit-anti-mouse IgG e.g. from DAKO, Glostrup, Denmark) can be added (e.g diluted 1:1000 in dilution buffer) and incubated for 30 minutes. The plates can then be washed in washing buffer, and a chromogenic substrate, e.g. OPD substrate (e.g. from Sigma-Aldrich, Vallensbæk Strand, Denmark) can be added. The reaction can be stopped with e.g 2N H₂SO₄ and the optical density be measured in e.g. a Dynex MRX ELISA plate reader at 490 nm. Serum antibody concentrations can be calculated e.g. by relating the optical densities of the samples to a standard curve. In order to generate the standard curve, a wide range of anti-CEA antibodies are available commercially and/or from academic sources.

The ELISA can be modified for different purposes, e.g. the reactivity against CEA peptides can be monitored using peptide ELISA for mapping of the fine specificity of anti-CEA antisera. Sandwich ELISA can be used to monitor whether the anti-CEA antisera contains reactivities that can displace e.g. a biotin-labeled, therapeutically relevant anti-CEA antibody from binding to plate-coated CEA. Specialized ELISA kits can be used to determine the isotype subclass distribution in anti-CEA etc.

Assays to Monitor Anti-CEA CTL Activity

It can be investigated whether immunization with CEA vaccines (DNA and/or protein-based vaccines) can induce a CEA-specific CTL response. CEA derived peptides (e.g. YLSGANLNL, YLSGADLNL, HLFGYSWYK, SYLSGANLNL, QYSWFVNGTF, TYACFVSNL) that are known to be able to bind to MHC class I can be synthesized and used to pulse antigen presenting cells in e.g. typical cytotoxic T cell assays. CEA expressing target cells (e.g. MHC class I expressing tumor cell lines transfected with CEA) can be used as targets for CEA-specific cytotoxic T lymphocytes (CTLs). Mice will be vaccinated with CEA-encoding DNA in vivo expression vectors and/or proteins using an appropriate vaccination scheme, cf. above. As positive control, mice could be immunized with e.g. MHC class I peptides derived from the CEA sequence. After a suitable time interval, e.g. three weeks after the first immunization, splenocytes from immunized mice can be re-stimulated e.g. with mytomicin C-treated (e.g. 50 μg/ml, 20 min at 37° C.) syngeneic splenocytes loaded with CEA peptides. For this re-stimulation process, a suitable number (e.g. 100×10⁶) of peptide-loaded syngeneic splenocytes can be mixed with a suitable number (e.g. 60×10⁶) of splenocytes from vaccinated mice in and incubated at 37° C. for e.g. 7 days. After the restimulation process, the cytotoxic activity of the effector cells can be monitored e.g. in a chromium release assay. A suitable number (e.g. 5×10⁶) of target cells is labeled with ⁵¹Chromium (e.g. 200 mCi), if necessary loaded with CEA-derived peptide, and used as targets in a ⁵¹Chromium release assay. Cell lines transfected with human CEA can also be used as targets for CTLs. Such CTL assays can e.g. be performed in normal wild type mice, human CEA transgenic mice and/or mice transgenic for human HLA class I molecules (e.g. HHD mice) using suitable compatible target cells and cells for restimulation.

The ability of a CEA vaccine to induce CEA-specific CTL responses can also be measured using other assay methods including CEA specific tetramer stainings and Elispot assays; all such methods are well-known to the person skilled in immunology.

CEA Tumor Models

To test the effect of CEA vaccination in tumor models, transgenic mice expressing human CEA or normal strains such as e.g. Balb/c, C57B1/6 or others could be obtained either from a commercial animal supplier or an academic or other collaborator. A transplantable tumor cell line expressing the CEA antigen can be used to create an animal model that can be used to monitor whether CEA vaccination can inhibit a CEA positive tumor challenge. The transplantable tumor cell line can either be a tumor cell line which is compatible with the mouse strain transfected with the human CEA sequence—or it can be a tumor cell line derived from a tumor dissected from CEA transgenic mice. The tumor model studies can be designed either as prevention or as treatment studies. In prevention studies, the mice are implanted with CEA expressing tumours after the course of immunization and the growth of the tumour is measured in a designated period. In treatment studies, mice are implanted with the CEA expressing tumours followed by single or multiple vaccinations with the CEA vaccines. Tumour growth can then be followed for a designated period.

One suitable such animal model is described in Eades-Perner AM et al. 1994, Cancer Res 54, 4169-4176.

Passive Transfer of Anti-CEA Antibodies

In order to determine whether antibodies induced by CEA vaccinations can inhibit growth of CEA expressing tumors, a passive transfer model can be used. Sera from mice vaccinated with modified CEA protein and/or DNA can be pooled and the CEA specific antibody concentration determined by ELISA. A suitable volume (e.g. 100 to 200 μl) of the pooled mouse serum can be injected into the peritoneum of naïve mice one day before subcutaneous challenge with a suitable number of CEA expressing tumor cells. The tumor cells can either be obtained from a commercial source or tumor cells transfected with the human CEA sequence. Control animals will be injected with non-specific mouse IgG (e.g. from Jackson ImmunoResearch Laboratories, Inc., West Grove, Pa.) or with serum from mice injected with an irrelevant antigen or adjuvant only. The mice can be immune deficient strains, e.g. nude mice or SCID mice, allowing transplantation with MHC incompatible tumor cell lines, e.g. human tumor cell lines expressing CEA. Tumour growth will then be monitored for a suitable time period, e.g. several weeks. Similar studies can be performed where the animal species which is immunized with CEA vaccines are not mice (e.g. rats or rabbits), and the protocol is adjusted accordingly.

The invention will now be further described by the following numbered paragraphs:

1. A method for inducing an immune response against autologous carcinoembryonic antigen (CEA) in an animal, including a human being, the method comprising effecting uptake and processing by antigen presenting cells (APCs) in the animal of at least one modified CEA polypeptide or of a nucleic acid encoding the modified CEA polypeptide or of a pharmaceutically acceptable microorganism or virus expressing the modified CEA polypeptide, said at least one modified CEA polypeptide comprising
at least about 80 CEA-derived amino acids, either in the form of at least about 80 consecutive CEA-derived amino acids or in the form of at least about 80 amino acids constituted of uninterrupted CEA-derived CTL epitopes, and
at least one first T helper epitope (T_{H} epitope) foreign to the animal,
   thereby inducing a CTL response and/or an antibody response that targets the autologous CEA.

2. The method according to paragraph 1, wherein the Modified CEA polypeptide comprises at least about 100 CEA derived amino acids.

3. The method according to paragraph 1 or 2, wherein at least one CEA-derived CTL epitope is presented by the APC in association with an MHC Class I molecule on the surface of the APC and/or wherein said at least one first foreign T_{H} epitope is presented by an APC in association with an MHC Class II molecule on the surface of the APC.

4. The method according to any one of the preceeding paragraphs, wherein the APC is a dendritic cell or a macrophage.

5. The method according to any one of the preceeding paragraphs, wherein at least one modified CEA polypeptide is in the form of one first analogue of CEA, said first analogue comprising a variation of the amino acid sequence of CEA, said variation containing CEA-derived CTL epitope(s) and the at least one first foreign T_{H} epitope.

6. The method according to paragraph 5, wherein the at least one first analogue contains a substantial fraction of known and predicted CTL epitopes from autologous CEA.

7. The method according to paragraph 6, wherein the substantial fraction of known and predicted CTL epitopes in the amino acid sequence of the analogue are recognized by at least 90% of the MHC-I haplotypes recognizing all known and predicted CTL epitopes in CEA.

8. The method according to any one of paragraphs 5-7, wherein substantially all known CTL epitopes of the autologous CEA are present in the first analogue and/or wherein substantially all predicted CTL epitopes of the autologous CEA are present in the at least first analogue.

9. The method according to any one of paragraphs 5-8, wherein the at least one first analogue further comprises at least one B-cell epitope of the autologous CEA, so that immunization of the animal with the first analogue induces production of antibodies in the animal against the autologous CEA.

10. The method according to any one of the preceeding paragraphs, wherein one modified CEA polypeptide is in the form of at least one second analogue of the autologous CEA, said second analogue containing at least one B-cell epitope of the autologous CEA, so that immunization of the animal with the second analogue induces production of antibodies against the autologous CEA.

11. The method according to paragraph 10, wherein at least one second foreign T_{H} epitope is included in the second analogue.

12. The method according to any one of paragraphs 6-11, wherein the first and/or second analogue(s) comprise(s) a substantial fraction of the B-cell epitopes of the autologous CEA.

13. The method according to any one of the preceeding paragraphs, wherein the modified CEA polypeptide substantially includes the amino acid sequence of at least one domain.

14. The method according to any one of the preceeding paragraphs, wherein the modified CEA polypeptide can be provided by subjecting CEA to amino acid substitution and/or deletion and/or insertion and/or addition.

15. The method according to any one of the preceeding paragraphs, wherein the modified CEA polypeptide comprises
at least one first moiety effecting targeting of the modified CEA polypeptide to an antigen presenting cell (APC), and/or
at least one second moiety stimulating the immune system, and/or
at least one third moiety optimising presentation of the modified CEA to the immune system.

16. The method according to any one of the preceeding paragraphs, wherein the modified CEA polypeptide includes duplication of at least one B-cell epitope or of at least one CTL epitope of the autologous CEA.

17. The method according to any one of the preceeding paragraphs, wherein the first and/or, where applicable, second foreign T_{H} epitope(s) is/are immunodominant and/or wherein the first and/or, where applicable, second foreign T_{H} epitope(s) is/are promiscuous.

18. The method according to any one of the preceeding paragraphs, wherein the modified CEA polypeptide is provided by introduction of a foreign T_{H} epitope that is introduced in any one of the following regions of CEA:
in the C-terminus,
in the N-terminus,
in the loop structures in any one of domains 1-7 as shown in FIG. 1, and
between any two adjacent domains of CEA.

19. The method according to paragraph 18, wherein the foreign T_{H} epitope is introduced in a manner selected from
an addition to the C- or N-terminus of mature CEA;
an insertion before any one of CEA amino acids 1, 38, 39, 40, 41, 42, 111, 148, 149, 150, 151, 203, 326, 327, 328, 329, 381, 418, 419, 420, 421, 464, 504, 505, 506, 507, 559, 596, 597, 598, and 643;
a substitution that includes deletion of any one or all of amino acids 38, 39, 40, and 41;
a substitution that includes deletion of any or all of amino acids 148, 149, and 150;
a substitution that includes deletion of any one or all of amino acids 326, 327, and 328;
a substitution that includes deletion of any or all of amino acids 418, 419, and 420;
a substitution that includes deletion of any or all of amino acids 504, 505, and 506; and
a substitution that includes deletion of any one or both of amino acids 596 and 597,
   wherein the amino acid numbering corresponds to that of SEQ ID NO: 2.

20. The method according to any one of the preceeding paragraphs wherein the C-terminal GPI-anchor of CEA is preserved in the modified CEA polypeptide.

21. The method according to any one of paragraphs 1-19, wherein the C-terminal GPI-anchor of CEA is removed.

22. The method according to any one of paragraphs 11-19, wherein foreign T_{H} epitope(s) is/are selected from a natural T_{H} epitope and an artificial MHC-II binding peptide sequence

23. The method according to paragraph 22, wherein the natural T-cell epitope is selected from a Tetanus toxoid epitope, a diphtheria toxoid epitope, an influenza virus hemagluttinin epitope, and a P. falciparum CS epitope.

24. The method according to any one of the preceeding paragraphs, wherein non-CEA derived components such as foreign T_{H} epitopes or first, second and third moieties as defined in paragraph 15 are present in the form of
side groups attached covalently or non-covalently to suitable chemical groups in the amino acid sequence of the autologous CEA or a subsequence thereof, and/or
fusion partners to the amino acid sequence derived from the autologous CEA.

25. The method according to paragraph 24, wherein
the first moiety is a substantially specific binding partner for an APC specific surface antigen such as a carbohydrate for which there is a receptor on the APC, e.g. mannan or mannose, or wherein the first moiety is a hapten,
the second moiety is a cytokine selected from interferon γ (IFN-γ), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF), or an effective part thereof; a heat-shock protein selected from heat shock protein 70 (HSP70), heat shock protein 90 (HSP90), heat shock cognate 70 (HSC70), glucose-regulated protein 94 (GRP94), and calreticulin (CRT), or an effective part thereof; or a hormone,
the third moiety is a lipid such as a palmitoyl group, a myristyl group, a farnesyl group, a geranyl-geranyl group, a GPI-anchor, and an N-acyl diglyceride group.

26. The method according to any one of the preceeding paragraphs, wherein the modified CEA polypeptide substantially preserves the 3-dimensional structure of at least one of CEA domains 1-7.

27. The method according to paragraph 26, wherein the 3-dimensional structures of at least 4 of CEA domains 1-7 is substantially preserved, preferably those of domains 1-4.

28. The method according to paragraph 27, wherein the 3-dimensional structures of all CEA domains are substantially preserved.

29. The method according to any one of the preceeding paragraphs, comprising administering, to the animal, an immunogenically effective amount of the at least one modified CEA polypeptide.

30. The method according to paragraph 29, wherein said modified CEA is formulated together with a pharmaceutically and immunologically acceptable carrier and/or vehicle and, optionally an adjuvant.

31. The method according to paragraph 30, wherein the adjuvant is selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant.

32. The method according to paragraph 31, wherein the cytokine is as defined as in paragraph 25, or an effective part thereof, wherein the toxin is selected from the group consisting of listeriolycin (LLO), Lipid A (MPL, L180.5/RalLPS), and heat-labile enterotoxin, wherein the mycobacterial derivative is selected from the group consisting of muramyl dipeptide, complete Freund's adjuvant, RIBI, and a diester of trehalose such as TDM and TDE, wherein the immune targeting adjuvant is selected from the group consisting of CD40 ligand, CD40 antibodies or specifically binding fragments thereof, mannose, a Fab fragment, and CTLA-4, wherein the oil formulation comprises squalene or incomplete Freund's adjuvant, wherein the polymer is selected from the group consisting of a carbohydrate such as dextran, PEG, starch, mannan, and mannose; a plastic polymer; and latex such as latex beads, wherein the saponin is Quillaja saponaria saponin, Quil A, and QS21, and wherein the particle comprises latex or dextran.

33. The method according to any one of paragraphs 29-32, which includes administration via a route selected from the oral route and the parenteral route.

34. The method according to any one of paragraphs 29-33, which includes at least one administration a year, such as at least 2, 3, 4, 5, 6, and 12 administrations a year.

35. The method according to any one of paragraphs 1-28, comprising administering, to the animal, a non-pathogenic microorganism or virus which is carrying a nucleic acid fragment encoding and expressing the at least one modified CEA polypeptide.

36. The method according to paragraph 35, wherein the non-pathogenic microorganism or virus is administered once to the animal.

37. The method according to any one of paragraphs 1-28, comprising administering, to the animal, at least one nucleic acid fragment which encodes and expresses the at least one modified CEA polypeptide.

38. The method according to paragraph 37, wherein the at least one nucleic acid fragment is selected from naked DNA, DNA formulated with charged or uncharged lipids, DNA formulated in liposomes, emulsified DNA, DNA included in a viral vector, DNA formulated with a transfection-facilitating protein or polypeptide, DNA formulated with a targeting protein or polypeptide, DNA formulated with a targeting carbohydrate, DNA formulated with Calcium precipitating agents, DNA coupled to an inert carrier molecule, and DNA formulated with an adjuvant.

39. The method according to paragraph 38, wherein the adjuvant is selected from the group consisting of the adjuvants defined in any one of paragraphs 31 or 32.

40. The method according to any of paragraphs 38 or 39, wherein the mode of administration is as defined in paragraph 33 or 34.

41. A modified human CEA polypeptide that is capable of inducing an immune response against autologous CEA in a human subject, comprising at least about 80 CEA-derived amino acids, either in the form of at least about 80 consecutive CEA-derived amino acids or in the form of at least about 80 amino acids constituted of uninterrupted CEA-derived CTL epitopes, and at least one first non-human T helper epitope (T_{H} epitope).

42. The modified human CEA polypeptide according to paragraph 41, wherein the at least one foreign T_{H} epitope is present as an insertion in the CEA amino acid sequence or as a substitution of part of the CEA amino acid sequence or as the result of deletion of part of the CEA amino acid sequence.

43. The modified human CEA according to paragraph 41 or 42, comprising at least 100 CEA derived amino acids.

44. The modified human CEA according to any one of paragraphs 41-43, which comprises at least one substantially preserved CEA domain.

45. The modified human CEA according to paragraph 44, which comprises at least 3 substantially preserved CEA domains, preferably domains 1-3.

46. The modified human CEA according to paragraph 45, comprising all 7 CEA domains in substantially preserved form.

47. The modified human CEA according to any one of paragraphs 41-46, wherein the at least one foreign T_{H} epitope is introduced in any one of the following regions of CEA:
between the C-terminal membrane anchor and domain 7
in the loop structures in any one of domains 1-7 as shown in FIG. 1, and
between any two adjacent loops in CEA.

48. An immunogenic composition which comprises, as an effective immunogenic agent the modified human CEA according to any one of paragraphs 41-47 in admixture with a pharmaceutically and immunologically acceptable carrier or vehicle, and optionally an adjuvant.

49. A nucleic acid fragment which encodes a modified CEA polypeptide according to any one of paragraphs 41-47.

50. A vector carrying the nucleic acid fragment according to paragraph 49.

51. The vector according to paragraph 50 being capable of autonomous replication.

52. The vector according to paragraph 50 or 51 being selected from the group consisting of a plasmid, a phage, a cosmid, a mini-chromosome, and a virus.

53. The vector according to any one of paragraphs 50-52, comprising, in the 5′→3′ direction and in operable linkage, a promoter for driving expression of the nucleic acid fragment according to paragraph 49, optionally a nucleic acid sequence encoding a leader peptide enabling secretion of or integration into the membrane of the polypeptide fragment, the nucleic acid fragment according to paragraph 49, and optionally a nucleic acid sequence encoding a terminator.

54. The vector according to any one of paragraphs 50-53 which, when introduced into a host cell, is integrated in the host cell genome or is not capable of being integrated in the host cell genome.

55. A transformed cell carrying the vector of any one of paragraphs 50-54.

56. A composition for inducing production of antibodies against CEA, the composition comprising
a nucleic acid fragment according to paragraph 49 or a vector according to any one of paragraphs 50-54, and
a pharmaceutically and immunologically acceptable diluent and/or vehicle and/or adjuvant.

57. A stable cell line which carries the vector according to any one of paragraphs 50-54 and which expresses the nucleic acid fragment according to paragraph 49, and which optionally secretes or carries the modified CEA according to any one of paragraphs 41-47 on its surface.

58. A method for the preparation of the cell line according to paragraph 57, the method comprising transforming a host cell with the nucleic acid fragment according to paragraph 49 or with the vector according to any one of paragraphs 50-54.

1. A method for inducing an immune response against autologous carcinoembryonic antigen (CEA) in an animal, including a human being, the method comprising effecting uptake and processing by antigen presenting cells (APCs) in the animal of at least one modified CEA polypeptide or of a nucleic acid encoding the modified CEA polypeptide or of a pharmaceutically acceptable microorganism or virus expressing the modified CEA polypeptide, said at least one modified CEA polypeptide comprising
at least about 80 CEA-derived amino acids, either in the form of at least about 80 consecutive CEA-derived amino acids or in the form of at least about 80 amino acids constituted of uninterrupted CEA-derived CTL epitopes, and
at least one first T helper epitope (T_{H} epitope) foreign to the animal, thereby inducing a CTL response and/or an antibody response that targets the autologous CEA.

2. The method according to claim 1, wherein the Modified CEA polypeptide comprises at least about 100 CEA derived amino acids.

3. The method according to claim 1, wherein at least one CEA-derived CTL epitope is presented by the APC in association with an MHC Class I molecule on the surface of the APC and/or wherein said at least one first foreign T_{H} epitope is presented by an APC in association with an MHC Class II molecule on the surface of the APC.

4. The method according to any claim 1, wherein the APC is a dendritic cell or a macrophage.

5. The method according to claim 1, wherein at least one modified CEA polypeptide is in the form of one first analogue of CEA, said first analogue comprising a variation of the amino acid sequence of CEA, said variation containing CEA-derived CTL epitope(s) and the at least one first foreign T_{H} epitope.

6. The method according to claim 5, wherein the at least one first analogue contains a substantial fraction of known and predicted CTL epitopes from autologous CEA.

7. The method according to claim 6, wherein the substantial fraction of known and predicted CTL epitopes in the amino acid sequence of the analogue are recognized by at least 90% of the MHC-I haplotypes recognizing all known and predicted CTL epitopes in CEA.

8. The method according to claim 5, wherein substantially all known CTL epitopes of the autologous CEA are present in the first analogue and/or wherein substantially all predicted CTL epitopes of the autologous CEA are present in the at least first analogue.

9. The method according to claim 5, wherein the at least one first analogue further comprises at least one B-cell epitope of the autologous CEA, so that immunization of the animal with the first analogue induces production of antibodies in the animal against the autologous CEA.

10. The method according to claim 1, wherein one modified CEA polypeptide is in the form of at least one second analogue of the autologous CEA, said second analogue containing at least one B-cell epitope of the autologous CEA, so that immunization of the animal with the second analogue induces production of antibodies against the autologous CEA.

11. The method according to claim 10, wherein at least one second foreign T_{H} epitope is included in the second analogue.

12. The method according to claim 6, wherein the first and/or second analogue(s) comprise(s) a substantial fraction of the B-cell epitopes of the autologous CEA.

13. The method according to claim 1, wherein the modified CEA polypeptide substantially includes the amino acid sequence of at least one domain.

14. The method according to claim 1, wherein the modified CEA polypeptide can be provided by subjecting CEA to amino acid substitution and/or deletion and/or insertion and/or addition.

15. The method according to claim 1, wherein the modified CEA polypeptide comprises
at least one first moiety effecting targeting of the modified CEA polypeptide to an antigen presenting cell (APC), and/or
at least one second moiety stimulating the immune system, and/or
at least one third moiety optimising presentation of the modified CEA to the immune system.

16. The method according to claim 1, wherein the modified CEA polypeptide includes duplication of at least one B-cell epitope or of at least one CTL epitope of the autologous CEA.

17. The method according to claim 1, wherein the first and/or, where applicable, second foreign T_{H} epitope(s) is/are immunodominant and/or wherein the first and/or, where applicable, second foreign T_{H} epitope(s) is/are promiscuous.

18. The method according to claim 1, wherein the modified CEA polypeptide is provided by introduction of a foreign T_{H} epitope that is introduced in any one of the following regions of CEA:
in the C-terminus,
in the N-terminus,
in the loop structures in any one of domains 1-7 as shown in FIG. 1, and
between any two adjacent domains of CEA.

19. The method according to claim 18, wherein the foreign T_{H} epitope is introduced in a manner selected from
an addition to the C- or N-terminus of mature CEA;
an insertion before any one of CEA amino acids 1, 38, 39, 40, 41, 42, 111, 148, 149, 150, 151, 203, 326, 327, 328, 329, 381, 418, 419, 420, 421, 464, 504, 505, 506, 507, 559, 596, 597, 598, and 643;
a substitution that includes deletion of any one or all of amino acids 38, 39, 40, and 41;
a substitution that includes deletion of any or all of amino acids 148, 149, and 150;
a substitution that includes deletion of any one or all of amino acids 326, 327, and 328;
a substitution that includes deletion of any or all of amino acids 418, 419, and 420;
a substitution that includes deletion of any or all of amino acids 504, 505, and 506; and
a substitution that includes deletion of any one or both of amino acids 596 and 597,
wherein the amino acid numbering corresponds to that of SEQ ID NO: 2.

20. The method according to claim 1 wherein the C-terminal GPI-anchor of CEA is preserved in the modified CEA polypeptide.

21. The method according to claim 1, wherein the C-terminal GPI-anchor of CEA is removed.

22. The method according to claim 11, wherein foreign T_{H} epitope(s) is/are selected from a natural T_{H} epitope and an artificial MHC-II binding peptide sequence

23. The method according to claim 22, wherein the natural T-cell epitope is selected from a Tetanus toxoid epitope, a diphtheria toxoid epitope, an influenza virus hemagluttinin epitope, and a P. falciparum CS epitope.

24. The method according to claim 15, wherein non-CEA derived components such as foreign T_{H} epitopes or first, second and third moieties are present in the form of
side groups attached covalently or non-covalently to suitable chemical groups in the amino acid sequence of the autologous CEA or a subsequence thereof, and/or
fusion partners to the amino acid sequence derived from the autologous CEA.

25. The method according to claim 24, wherein
the first moiety is a substantially specific binding partner for an APC specific surface antigen such as a carbohydrate for which there is a receptor on the APC, e.g. mannan or mannose, or wherein the first moiety is a hapten,
the second moiety is a cytokine selected from interferon γ (IFN-γ), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF), or an effective part thereof; a heat-shock protein selected from heat shock protein 70 (HSP70), heat shock protein 90 (HSP90), heat shock cognate 70 (HSC70), glucose-regulated protein 94 (GRP94), and calreticulin (CRT), or an effective part thereof; or a hormone,
the third moiety is a lipid such as a palmitoyl group, a myristyl group, a farnesyl group, a geranyl-geranyl group, a GPI-anchor, and an N-acyl diglyceride group.

26. The method according to claim 1, wherein the modified CEA polypeptide substantially preserves the 3-dimensional structure of at least one of CEA domains 1-7.

27. The method according to claim 26, wherein the 3-dimensional structures of at least 4 of CEA domains 1-7 is substantially preserved.

28. The method of claim 27, wherein domains 1-4 are substantially preserved.

29. The method according to claim 27, wherein the 3-dimensional structures of all CEA domains are substantially preserved.

30. The method according to claim 1, comprising administering, to the animal, an immunogenically effective amount of the at least one modified CEA polypeptide.

31. The method according to claim 30, wherein said modified CEA is formulated together with a pharmaceutically and immunologically acceptable carrier and/or vehicle and, optionally an adjuvant.

32. The method according to claim 31, wherein the adjuvant is selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant.

33. The method according to claim 32, wherein the cytokine is selected from interferon γ (IFN-γ), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF), or an effective part thereof; a heat-shock protein selected from heat shock protein 70 (HSP70), heat shock protein 90 (HSP90), heat shock cognate 70 (HSC70), glucose-regulated protein 94 (GRP94), and calreticulin (CRT), or an effective part thereof; or a hormone, or an effective part thereof, wherein the toxin is selected from the group consisting of listeriolycin (LLO), Lipid A (MPL, L180.5/RalLPS), and heat-labile enterotoxin, wherein the mycobacterial derivative is selected from the group consisting of muramyl dipeptide, complete Freund's adjuvant, RIBI, and a diester of trehalose such as TDM and TDE, wherein the immune targeting adjuvant is selected from the group consisting of CD40 ligand, CD40 antibodies or specifically binding fragments thereof, mannose, a Fab fragment, and CTLA-4, wherein the oil formulation comprises squalene or incomplete Freund's adjuvant, wherein the polymer is selected from the group consisting of a carbohydrate such as dextran, PEG, starch, mannan, and mannose; a plastic polymer; and latex such as latex beads, wherein the saponin is Quillaja saponaria saponin, Quil A, and QS21, and wherein the particle comprises latex or dextran.

34. The method according to claim 30, which includes administration via a route selected from the oral route and the parenteral route.

35. The method according to claim 30, which includes at least one administration per year.

36. The method of claim 35, wherein there are at least 2, 3, 4, 5, 6, or 12 administrations per year.

37. The method according to claim 1, comprising administering, to the animal, a non-pathogenic microorganism or virus which is carrying a nucleic acid fragment encoding and expressing the at least one modified CEA polypeptide.

38. The method according to claim 37, wherein the non-pathogenic microorganism or virus is administered once to the animal.

39. The method according to claim 1, comprising administering, to the animal, at least one nucleic acid fragment which encodes and expresses the at least one modified CEA polypeptide.

40. The method according to claim 39, wherein the at least one nucleic acid fragment is selected from naked DNA, DNA formulated with charged or uncharged lipids, DNA formulated in liposomes, emulsified DNA, DNA included in a viral vector, DNA formulated with a transfection-facilitating protein or polypeptide, DNA formulated with a targeting protein or polypeptide, DNA formulated with a targeting carbohydrate, DNA formulated with Calcium precipitating agents, DNA coupled to an inert carrier molecule, and DNA formulated with an adjuvant.

41. The method according to claim 40, wherein the adjuvant is selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant.

42. The method according to claim 40, wherein the mode of administration is via a route selected from the oral route and the parenteral route.

43. A modified human CEA polypeptide that is capable of inducing an immune response against autologous CEA in a human subject, comprising at least about 80 CEA-derived amino acids, either in the form of at least about 80 consecutive CEA-derived amino acids or in the form of at least about 80 amino acids constituted of uninterrupted CEA-derived CTL epitopes, and at least one first non-human T helper epitope (T_{H} epitope).

44. The modified human CEA polypeptide according to claim 43, wherein the at least one foreign T_{H} epitope is present as an insertion in the CEA amino acid sequence or as a substitution of part of the CEA amino acid sequence or as the result of deletion of part of the CEA amino acid sequence.

45. The modified human CEA according to claim 43, comprising at least 100 CEA derived amino acids.

46. The modified human CEA according to claim 43, which comprises at least one substantially preserved CEA domain.

47. The modified human CEA according to claim 46, which comprises at least 3 substantially preserved CEA domains.

48. The modified human CEA according to claim 47, comprising all 7 CEA domains in substantially preserved form.

49. The modified human CEA according to claim 43, wherein the at least one foreign T_{H} epitope is introduced in any one of the following regions of CEA:
between the C-terminal membrane anchor and domain 7
in the loop structures in any one of domains 1-7 as shown in FIG. 1, and
between any two adjacent loops in CEA.

50. An immunogenic composition which comprises, as an effective immunogenic agent the modified human CEA according to claim 43 in admixture with a pharmaceutically and immunologically acceptable carrier or vehicle, and optionally an adjuvant.

51. A nucleic acid fragment which encodes a modified CEA polypeptide according to claim 43.

52. A vector carrying the nucleic acid fragment according to claim 51.

53. The vector according to claim 52 being capable of autonomous replication.

54. The vector according to claim 52 being selected from the group consisting of a plasmid, a phage, a cosmid, a mini-chromosome, and a virus.

55. The vector according to claim 52, comprising, in the 5′→3′ direction and in operable linkage, a promoter for driving expression of the nucleic acid fragment which encodes a modified CEA polypeptide that is capable of inducing an immune response against autologous CEA in a human subject, comprising at least about 80 CEA-derived amino acids, either in the form of at least about 80 consecutive CEA-derived amino acids or in the form of at least about 80 amino acids constituted of uninterrupted CEA-derived CTL epitopes, and at least one first non-human T helper epitope (T_{H} epitope), optionally a nucleic acid sequence encoding a leader peptide enabling secretion of or integration into the membrane of the polypeptide fragment, the nucleic acid fragment which encodes a modified CEA polypeptide, and optionally a nucleic acid sequence encoding a terminator.

56. The vector according to claim 52 which, when introduced into a host cell, is integrated in the host cell genome or is not capable of being integrated in the host cell genome.

57. A transformed cell carrying the vector of claim 52.

58. A composition for inducing production of antibodies against CEA, the composition comprising
a nucleic acid fragment which encodes a modified CEA polypeptide that is capable of inducing an immune response against autologous CEA in a human subject, comprising at least about 80 CEA-derived amino acids, either in the form of at least about 80 consecutive CEA-derived amino acids or in the form of at least about 80 amino acids constituted of uninterrupted CEA-derived CTL epitopes, and at least one first non-human T helper epitope (T_{H} epitope), and
a pharmaceutically and immunologically acceptable diluent and/or vehicle and/or adjuvant.

59. A stable cell line which carries the vector according to claim 52 and which expresses the nucleic acid fragment which encodes a modified CEA polypeptide that is capable of inducing an immune response against autologous CEA in a human subject, comprising at least about 80 CEA-derived amino acids, either in the form of at least about 80 consecutive CEA-derived amino acids or in the form of at least about 80 amino acids constituted of uninterrupted CEA-derived CTL epitopes, and at least one first non-human T helper epitope (T_{H} epitope), and which optionally secretes or carries the modified CEA on its surface.

60. A method for the preparation of the cell line according to claim 59, the method comprising transforming a host cell with the nucleic acid fragment that encodes a modified CEA polypeptide that is capable of inducing an immune response against autologous CEA in a human subject, comprising at least about 80 CEA-derived amino acids, either in the form of at least about 80 consecutive CEA-derived amino acids or in the form of at least about 80 amino acids constituted of uninterrupted CEA-derived CTL epitopes, and at least one first non-human T helper epitope (T_{H} epitope), or with a vector carrying the nucleic acid fragment that encodes the modified CEA polypeptide.
